(19) United States
(12) Patent Application Publication
(10) Pub. No.: US 2011/0098556 A1
(43) Pub. Date: Apr. 28, 2011

(54) Computer-Based Method And System For Imaging-Based Dynamic Function Evaluation Of An Organ

(76) Inventors: Lennart Blomqvist, Taby (SE); Anders Nordell, Taby (SE); Eduard Jonas, Taby (SE); Henrik Nilsson, Taby (SE)

(21) Appl. No.: 12/922,152

(22) PCT Filed: Mar. 11, 2009
(86) PCT No.: PCT/EP09/52888

§ 371 Date: Jan. 13, 2011

Related U.S. Application Data
(60) Provisional application No. 61/035,683, filed on Mar. 11, 2008.

(51) Int. Cl. A61B 5/055

(52) U.S. Cl. 600/419; 600/410; 600/420

(57) ABSTRACT

A computer-based method of determining a functional assessment of at least one organ having secretional or excretional functions, such as a liver or kidneys, of a human is disclosed. The method comprises processing a four-dimensional (4D) image data set of said human comprising data for an assessment of said organ function, wherein said 4D image data is acquired by an image modality; and wherein said processing said 4D image data comprises performing a deconvolutional analysis (DA) comprising a matrix inversion using singular value decomposition (SVD) based on said 4D image data.

### FIELD OF THE INVENTION

This invention pertains in general to the field of imaging-based organ function assessment. More particularly the invention relates to a method and system for imaging-based dynamic function evaluation of at least one organ having secretional or excretional functions, such as a liver and/or kidneys of a human, and related methods and use thereof. Even more particularly some embodiments of the invention pertain to Magnetic resonance imaging (MRI)-based dynamic functional assessment of at least one organ having secretional or excretional functions, in particular hepatic and renal function assessment taking advantage of organ specific contrast enhancement agents, such as Gd-EOB-DTPA.

### BACKGROUND OF THE INVENTION

The assessment of liver function currently relies mostly on serum analyte measurements, scoring models such as Child-Pugh and MELD and to some extent, clearance tests. The simplicity and low cost of analyte measurement accounts for their frequent use in clinical practice. They give indirect information about the cellular integrity of the hepatocytes, and their synthetic and secretory function, but the sensitivity and specificity of analyte measurements are generally considered to be low. Furthermore there is often a significant delay between impairment in hepatic function and a detectable change in serum levels of analytes. Clearance tests measure the rate at which a test substrate is cleared from the bloodstream, and in some cases the formation of a metabolite. Various test substrates have been used, such as bromosulphthalein (BSP), galactose and indocyanine green (ICG). Some clearance rates are highly dependent on hepatic perfusion, which has been shown to undergo significant change in liver disease such as malignancy and end-state cirrhosis. Clearance tests and analyte measurements are indicators of global liver function, and cannot detect deterioration in hepatocyte function or bile excretion on a segmental or regional level. Clearance tests are cumbersome and are generally seldom used in clinical practice.

In addition, organ function, such as liver function, has previously been assessed based on input data from single photon emission computed tomography (SPECT). However, this application has not gained a widespread clinical use due to implementation reasons and patient dosage limitations, amongst others. Scintigraphic methods are currently the only choice for imaging-based liver function testing in clinical use. A radioactive tracer, most commonly from the 99mTc-IDA-family, is injected into the bloodstream and the tracer activity in a region of interest (ROI) placed over the liver is sampled over time, i.e. a dynamic study is performed. The activity in the blood pool is registered from a ROI placed over the heart and/or the spleen, and is often used to define an input function.

However, scintigraphic methods are hampered by a number of drawbacks, such as the low resolution and limited anatomic detail in the images obtained. In the liver, regional differences in hepatocyte function may therefore be hard or impossible to detect.

In scintigraphic studies, measurement of hepatic function has been assessed either from semi-quantitative analyses of hepatic activity curves, using parameters such as excretion half-time (t½), time to peak (TTP) and maximum activity (Cmax), also known as summary parameters, or calculating hepatic extraction fraction (HEF) or mean transit time (MTT). The results of the summary parameters are, however, to be considered with caution. For instance, the tissue concentration or activity-over-time-curve in any perfusion study is highly dependent on differences in input function (IF) and tissue residue functions between different patients or studies.

Thus, there is a need for a new or at least improved method and/or system for dynamic function evaluation of an organ, which preferably is imaging-based.

Hence, a new or at least improved method and/or system for imaging-based dynamic function evaluation of an organ having a secretional or excretional function, such as the liver, would be advantageous. In particular, the new or improved method is desired to be flexible, cost-effective, comfortable for patients, safe, and/or compatible with existing drugs and medical procedures.

### SUMMARY OF THE INVENTION

Accordingly, embodiments of the present invention preferably seek to mitigate, alleviate or eliminate one or more deficiencies, disadvantages or issues in the art, such as the above-identified, singly or in any combination by providing a system, a method, a computer program, a medical workstation, and a medical method according to the appended patent claims.

According to a first aspect of the invention, a computer-based system adapted to determine a function over time of at least one organ of a human is provided. The organ is an organ that has a secretional or excretional function, such as a liver and/or kidneys. The system comprises a processing unit configured to process a set of four-dimensional (4D) image data acquired by an image modality, and configured to determine a value of a parameter related to the function of the at least one organ per volume unit of the at least one organ based on the set of four-dimensional (4D) image data, whereby a diagnosis of a dysfunction of the organ is facilitated by a comparison of the determined value of the parameter with previously determined values of the parameters of a healthy population.

According to a second aspect of the invention, a computer program storable on a computer readable medium, for processing by a computing device for determining a function over time of at least one secretional or excretional organ, such as a liver and/or kidneys of a human, is provided. The computer program comprises a plurality of code segments, comprising a first code segment for determining a value of a parameter related to the function of the at least one organ per volume unit of the at least one organ based on processing of a set of four-dimensional (4D) image data of the human acquired by an image modality, facilitating diagnosis of a dysfunction of the organ by a comparison of the determined value of the parameter with previously determined values of the parameters of a healthy population.

According to a third aspect of the invention, a computer-implemented method of determining a function over time of at least one secretional or excretional organ, such as a liver and/or kidneys of a human, is provided. Determining the function of the at least one organ comprises determining a value of a parameter related to the function of the at least one organ per volume unit of the at least one organ, and wherein determining the function is based on processing of a set of four-dimensional (4D) image data of the human acquired by an image modality, facilitating diagnosis of a dysfunction of the organ by a comparison of the determined value of the parameter with previously determined values of the parameters of a healthy population.

According to a fourth aspect of the invention, a graphical user interface is provided. The graphical user interface comprises a result of the method of the third aspect of the invention, comprising HEF, or irBF, or HEF and irBF, in the form of at least one parametric map.

According to a fifth aspect of the invention, a method of computer-based virtual planning of a surgical procedure comprising the method of the third aspect of the invention is provided.

According to a sixth aspect of the invention, a medical workstation comprised in the system of the first aspect of the invention is provided, for executing said computer program of said second aspect of the invention.

Further embodiments of the invention are defined in the dependent claims, wherein features for the second and subsequent aspects of the invention are as for the first aspect mutatis mutandis.

Embodiments are based on the use of image data provided from an image modality. The image modality is advantageously providing image data of a body suitable for examining an uptake of a contrast agent in an organ.

Some embodiments are based on the use of organ specific contrast agents to enhance contrast of image data of the secretional or excretional organ.

Some embodiments are based on the use of a paramagnetic contrast agent, such as a gadolinium compound. Gadolinium-enhanced tissues and vascular structures appear extremely bright on T1-weighted MRI images. This provides high sensitivity for detection of e.g. vascular tissues and permits assessment of organ perfusion and may provide an assessment of the organ's function, e.g. of the liver's function. When using a hepatocyte-specific contrast agent, some embodiments are based on dynamic hepatocyte-specific contrast enhanced (DHCE) MRI, DHCE-MRI. When using a hepato-renal-specific contrast agent, some embodiments are based on dynamic hepato-renal specific contrast enhanced (DHRCE) MRI, DHRCE-MRI.

Embodiments of the present method and/or system have a potentially important impact on the possibility to depict hepatic function regionally, which may be useful in identifying regional liver disease and in monitoring response to pharmacological therapy and surgical or endoscopic interventions.

Some embodiments of the invention provide for organ function assessment independent of the type of contrast agent used.

Some embodiments of the invention provide for organ function assessment independent of the type of pulse sequence of an MRI modality used.

Some embodiments of the invention provide for assessment of organ function on a segmental or sub-segmental level thereof.

Some embodiments provide for simultaneous determination of a function of more than one organ at the same time, for instance of a liver and kidneys. In this way a synergetic determination of a physiological function of these organs and an inter-relationship of their function is facilitated. For instance waste products from the liver are carried by the blood to the kidneys. The kidneys filter out these waste products and expel them from the body in urine. Diagnosis of dysfunctions in this delicate organ interaction are thus provided.

Some embodiments provide for identification of segments or sub-segments of organs that are in dysfunction. This in turn provides for facilitating virtual planning of surgical procedures to treat the dysfunction.

Some embodiments of the invention provide for diagnostic assessment of liver function in primary biliary cirrhosis (PBC).

Some embodiments of the invention provide for diagnostic assessment of liver function in primary sclerosing cholangitis (PSC).

Some embodiments provide for diagnosis of a dysfunction of a secretional or excretional organ by comparison of measured or determined parameters and comparison with previously determined values of such parameters of a healthy population.

The term “function” of an organ relates to its physiological operation or action. For instance, the secretional or excretional function of secretional or excretional organs, such as the liver or kidneys, is determined by embodiments.

Embodiments are different from nuclear medicine, which is not comprised in the embodiments but expressively excluded from the latter. Radioactive tracers are not included in embodiments when referring to contrast agents or tracers in the detailed specification. The embodiments differ substantially, as scintigraphic practice is not able to provide segment specific functional analysis of organs. This is elucidated further below.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other aspects, features and advantages of which embodiments of the invention are capable will be apparent and elucidated from the following description of embodiments of the present invention, reference being made to the accompanying drawings, in which

FIG. 1 is a schematic drawing illustrating an image visualizing data acquired by an MRI modality showing a slice through an abdomen;

FIG. 2A is a schematic drawing illustrating an impulse function convoluted with an impulse response thereof;

FIG. 2B is a schematic drawing illustrating a non-ideal input function convoluted with an impulse response;

FIG. 3 is a graph illustrating a deconvoluted hepatic extraction (HE) curve, and a hepatic retention curve (HRC);

FIG. 4 is a schematic drawing illustrating the obtainment of a hepatic extraction curve;

FIG. 5 is a flow chart illustrating a method comprising an embodiment;

FIG. 6 is a schematic illustration of a portion of the method of FIG. 5;

FIG. 7 is a schematic illustration of a calculation portion of the method of FIG. 5;

FIG. 8 is a schematic illustration of a sectionized hepatic function assessment;

FIG. 9 is a graph illustrating a mean error and error bars for different simulated calculation methods;

FIGS. 10A to 10D are images based on data acquired by MRI and subsequent image processing based on different calculation methods;

FIG. 11 is a graph illustrating the result of calculating HEF from 4D image data comparing a Fourier Analysis and truncated singular value decomposition (TSVD) calculation thereof;

FIG. 12 is a schematic illustration of a system of an embodiment;

FIG. 13 is a schematic illustration of a computer program of an embodiment;

FIGS. 14A and 14B are graphs illustrating an overall distribution of HEF and RBF when DA with TSVD is compared to FA+tail;

FIGS. 15A and 15B are graphs illustrating distributions of HEF and RBF on a segmental level using both TSVD and FA+tail;

FIG. 16 is a schematic illustration of a compartmental model;

FIG. 17 is a graph illustrating the convergence of out-functions compared to measured parenchymal response functions;

FIG. 18 is a graph illustrating HEF-results from patients with morphological evidence of cirrhosis compared with the results from healthy controls presented on a segmental level;

FIG. 19 is a graph illustrating area-under-curve (AUC) results from patients with morphological evidence of cirrhosis compared with the results from the healthy controls presented on a segmental level;

FIG. 20 is a graph illustrating the pharmacokinetic parameter k₂₁ on a segmental level;

FIG. 21 is a graph illustrating the pharmacokinetic parameter k₃ on a segmental level;

FIG. 22 is a graph illustrating HEF presented on a segmental level;

FIG. 23 is a graph illustrating quantitatively assessed AUC presented on a segmental level;

FIG. 24 is a graph illustrating the pharmacokinetic transfer constant k₂₁ presented on a segmental level;

FIG. 25 is a graph illustrating the pharmacokinetic transfer constant k₃ presented on a segmental level;

FIGS. 26A and 26B are graphs illustrating parenchymal response curves, both from a PSC patient and for a segment in healthy volunteers; and

FIG. 27 is a schematic illustration of calculating local HEF and local irBF with compensation for partial volume effects.

The terminology used in the detailed description of the embodiments illustrated in the accompanying drawings is not intended to be limiting of the invention. In the drawings, like numbers refer to like elements.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. In more detail, some embodiments given in the following description focus on a method and system applicable to a liver function assessment and in particular to an MRI-based image analysis for liver function assessment. However, it will be appreciated that the invention is not limited to this application, but may be applied to many other medical areas, procedures and/or secretional or excretional organs, including for example those mentioned further below.

In an embodiment, T1-weighted dynamic hepatic specific contrast enhanced (DHCE) MRI provides 3D image data. A plurality of 3D data sets acquired at subsequent times provide for a 4D image data set. The 4D image data set is processed for an assessment of liver function on a segmental or sub-segmental level of the liver. The segment may be as small as down to a voxel level of the 4D image data. A value for a parameter for the liver function is determined from processing the 4D data set.

The blood flow in the liver is determined on a segmental or sub-segmental level relative to the input blood flow of the liver (input relative Blood Flow, irBF).

The blood flow in the liver is determined on a segmental or sub-segmental level relative to the venous blood flow in the liver.

The blood flow in the liver may be determined on the whole liver. Alternatively or in addition, the blood flow may be determined on a segmental or sub-segmental level. The blood flow may be determined relative to the arterial blood flow in the liver.

The Hepatic Extraction Fraction (HEF) of the liver is in some embodiments determined on a segmental or sub-segmental level, down to a voxel level. The HEF has previously only been determined on a level of the entire organ. As the HEF is provided on a segmental or sub-segmental level, down to a voxel level, novel and more effective opportunities of diagnosis and treatment arise.

The function of the liver may be determined per volume unit thereof. The volume of the liver or segments thereof may be determined from the 3D image data provided by the image modality, e.g. the MRI modality. In this manner the local HEF may be correlated to a specific volume of the liver, i.e. the HEF/volume is determined.

This allows for instance, a virtual planning of surgical procedures to be made. Computer-based virtual planning of surgical removal of a part of an organ before resecting a diseased part of the liver may allow calculation of residual liver function after surgery. This is a major advantage both from a clinician's view and with regard to patient safety.

The Hepatic Extraction Fraction (HEF) and/or input relative Blood Flow (irBF) of the liver are in some embodiments, on a segmental or sub-segmental organ level, determined based on truncated singular value decomposition (TSVD) calculations. This is for instance computationally advantageous, allowing clinically acceptable calculation times.

TSVD is in some embodiments advantageously used for determining parametric maps. The parametric maps provide for an efficient and quick diagnosis of an organ function.

In some embodiments HEF and irBF are visualized in the form of parametric maps.

In some embodiments HEF and irBF are visualized in the form of parametric maps superimposed on anatomical images.

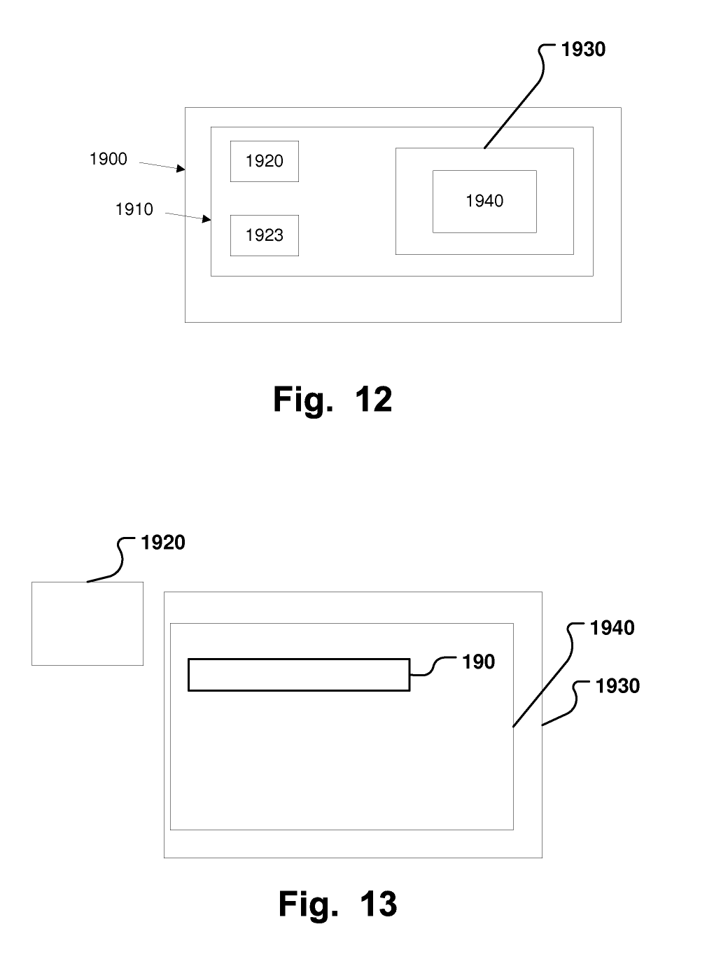

Calculation results, virtual planning of surgical procedures or other treatments may for instance be presented on a display of a medical workstation. Planning of a procedure or treatment based on calculation results of embodiments may be made visually on the display of a medical workstation, e.g. of the system described below with reference to FIG. 12, in an interactive way manipulated by user input.

In an embodiment, a dynamic liver function test using Magnetic Resonance Imaging (MRI) and Gadolinium ethoxybenzyl diethylenetriaminepentaacetic acid (Gd-EOB-DTPA (Primovist®, Schering AG, Berlin, Germany)) as a liver specific contrast agent is used. Gd-EOB-DTPA has unique properties of equal elimination through hepatic and renal pathways.

The dual pathway of uptake and excretion of the contrast agent (50% hepatocyte uptake, biliary excretion) (50% renal elimination by glomerular filtration) for Gd-EOB-DTPA allows the present model to be used for simultaneous monitoring and/or determination of both liver and renal function which is a unique property of the present method together with this contrast agent. Thus, Dynamic Hepato-Renal Specific Contrast Enhanced (DHRCE) Magnetic Resonance Imaging (MRI) is provided and used for acquiring 3D or 4D images in or for embodiments.

Other gadolinium-based contrast agents available for clinical use and suitable for some aspects of the present method, comprise: Magnevist® Gadopentate dimeglumine of Bayer Schering Pharma; Omniscan® Gadodiamide of GE Healthcare; Dotarem®, Gd-DOTA of Gothia/Guerbet; ProHance®, Gadoteridol of Initios Medical AB/Bracco; Gadovist®, Gadobutrol of Bayer Schering Pharma.

Other tissue specific contrast agents available for clinical use and suitable for other aspects of the present method, comprise: Endorem®, (SPIO) (80-150 nm) Ferrumoxid of Gothia/Guerbet, Resovist®, (SPIO) (60 nm) ferucarbotran of Bayer Schering Pharma; Teslascan® Mangafodipir trisodium of GE Healthcare; MultiHance®, Gadobenate dimeglumine of Initios Medical AB/Bracco, Vasovist®, Gadofosvesettrinatrium of Bayer Schering Pharma. However, the latter contrast agents may not be suited for hepatocyte specific contrast enhancement, but for contrast enhancement of other secretional or excretional organs, such as kidneys.

Embodiments of the present method and system are not limited to the use of Gd-EOB-DTPA as contrast agent. Other future or currently available liver- or organ specific contrast agents may be suitable as well.

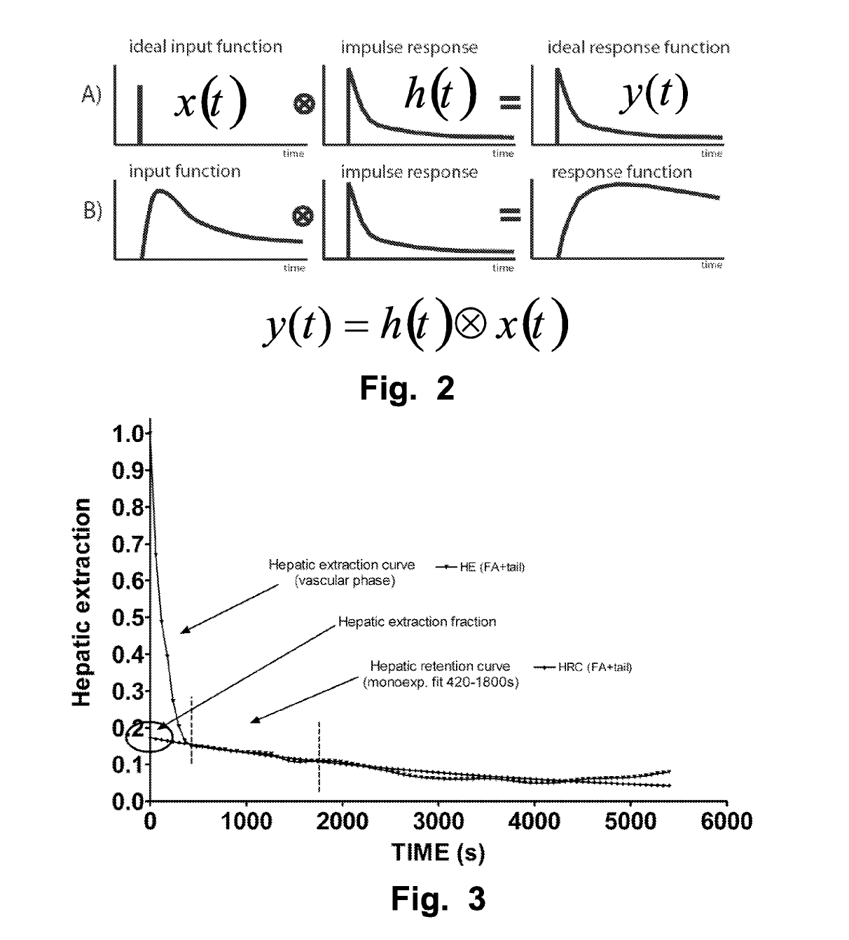

When contrast agents are intravenously administered, the concentration in the liver is affected by the blood recirculation and the dispersion over time. Therefore, the response function can be described as a convolution between the impulse response and the input function. FIG. 2A shows the ideal case, when the organ of interest is presented by a short impulse function, giving the impulse response. As mentioned, in reality the organ of interest is presented by an input function, which varies over time and will therefore influence the response function, as shown in FIG. 2B.

To overcome the effect of tracer recirculation Deconvolutional Analysis (DA) is applied using an afferent vascular relative enhancement curve as input function and a liver relative enhancement curve as the response function. For DA, matrix inversion and Singular Value Decomposition (SVD) is performed.

Measured hepatic contrast agent enhancement may be more dependent on hepatic perfusion than the actual hepatocellular function, making provision for input function imperative. This is especially true for tracers with a high hepatic extraction ratio.

Ideally, to overcome the effects of recirculation, administration of the tracer should be provided as a short intravascular bolus directly into the afferent blood supply of the liver, i.e. the portal vein or the hepatic artery. A peripheral intravenous administration, as used in clinical practice, will present the liver with only a small percentage of injected tracer during the first pass, equivalent to the cardiac output fraction received by the liver. Subsequently, the liver will constantly be presented with a changing concentration of tracer due to recirculation and simultaneous extraction and excretion. Administration of a tracer directly into the portal vein or hepatic artery is not conducted in a clinical situation when liver imaging is performed.

The principle, however, can be simulated by the use of deconvolutional analysis (DA). DA corrects an organ's time-activity curve for the changing concentrations of contrast agent being presented to the organ. The method has been validated in animal studies using deconvolution based on Fourier transforms (FT). FT is the most widely used deconvolution model in scintigraphic practice.

Deconvolutional analysis has hitherto not been used for determining a function of an organ per volume unit. This is in particular the case for secretional or excretional organs that are not simply perfused by blood, but have a secretional or excretional function in addition.

Matrix inversion using singular value decomposition (SVD), is a more advantageous mathematical model for DA but has not been used hitherto for determining liver function from image data.

The use of a hepatocyte-specific contrast agent, such as Gadolinium ethoxybenzyl diethylenetriaminepentaacetic acid ((Gd-EOB-DTPA), Primovist®, Bayer Schering Pharma AG, Berlin) has been shown to improve detection and characterization of focal liver lesions when used in T1-weighted MRI. The pharmacodynamic properties of Gd-EOB-DTPA are similar to those of the 99mTc-IDA-family with a hepatocellular uptake through the organic anionic transport system (OATS) and subsequent biliary excretion by glutathione-S-transferase. Pharmacokinetic studies show that about 50% of the administered dose of Gd-EOB-DTPA is extracted by the liver and secreted through the hepato-biliary pathway. The remaining 50% is eliminated by renal excretion. Thus, hepatic uptake of Gd-EOB-DTPA and subsequent T1-relaxation shortening are dependent on the integrity of the hepatocyte mass. Dynamic Gd-EOB-DTPA MRI has previously been used in animal models for the evaluation of hepatic function and dysfunction in various experimental settings, either using summary parameters or DA.

The pharmacodynamic properties of Gd-EOB-DTPA in combination with the high resolution obtained in MRI opens up the advantageous use of DHCE-MRI with Gd-EOB-DTPA as an imaging-based liver function test, providing the discriminating difference in function on a regional and/or even segmental level. This has not been provided in humans before.

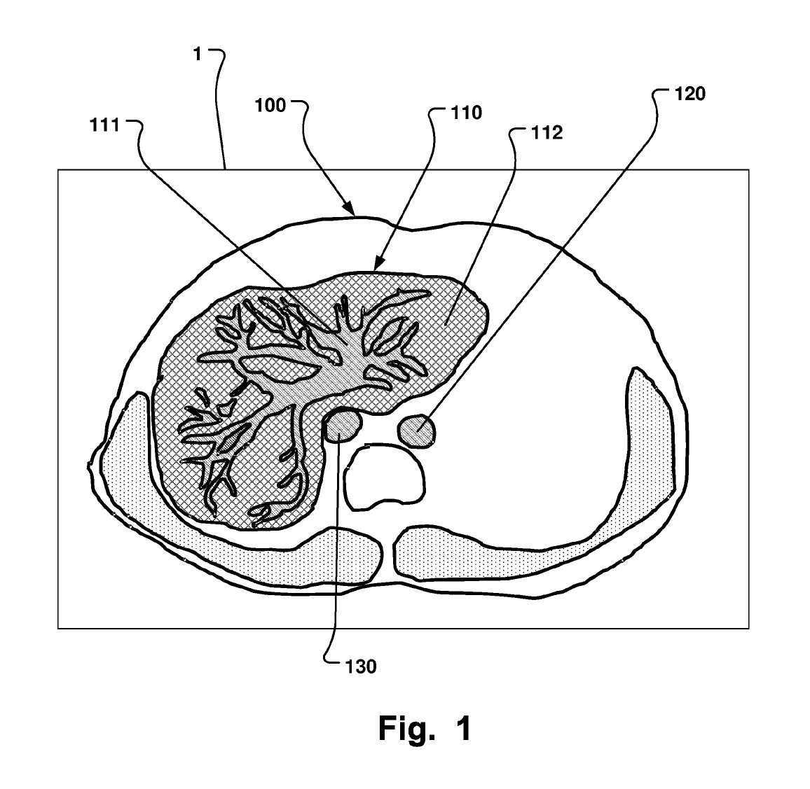

FIG. 1 is a schematic drawing illustrating an image 1 visualizing three-dimensional (3D) data acquired by an MRI modality showing a slice through an abdomen 100. The liver 110 is shown including parenchyma 112 (functional parts of the liver) and the portal portalpeddicles including bile ducts, portal vein branches and hepatic artery branches 111. Also illustrated are the inferior vena cava (IVC) 130 (in other sections even the hepatic veins draining from the liver into the IVC can be visualized) and the aorta 120.

FIG. 2A is a schematic drawing illustrating an impulse function convoluted with an impulse response thereof, and FIG. 2B is a schematic drawing illustrating a non-ideal input function convoluted with an impulse response; as mentioned above and explained in more detail below.

FIG. 3 is a graph illustrating a deconvoluted hepatic extraction (HE) curve, and a hepatic retention curve (HRC). The deconvoluted hepatic extraction (HE) curve (i.e. the impulse response) and the hepatic retention curve (HRC), which is a monoexponential fit to the time points in the HE curve between 420 and 1800 seconds, are shown in FIG. 3. The ratio between the peak value of the HE curve and the Y-axis intercept of the HRC is defined as the hepatic extraction fraction (HEF). In this case, Fourier analysis (FA+tail) was used for DA, and the HEF in the regarded example shown in FIG. 3 is approximately 17%. FIG. 3 will be elucidated in more detail below.

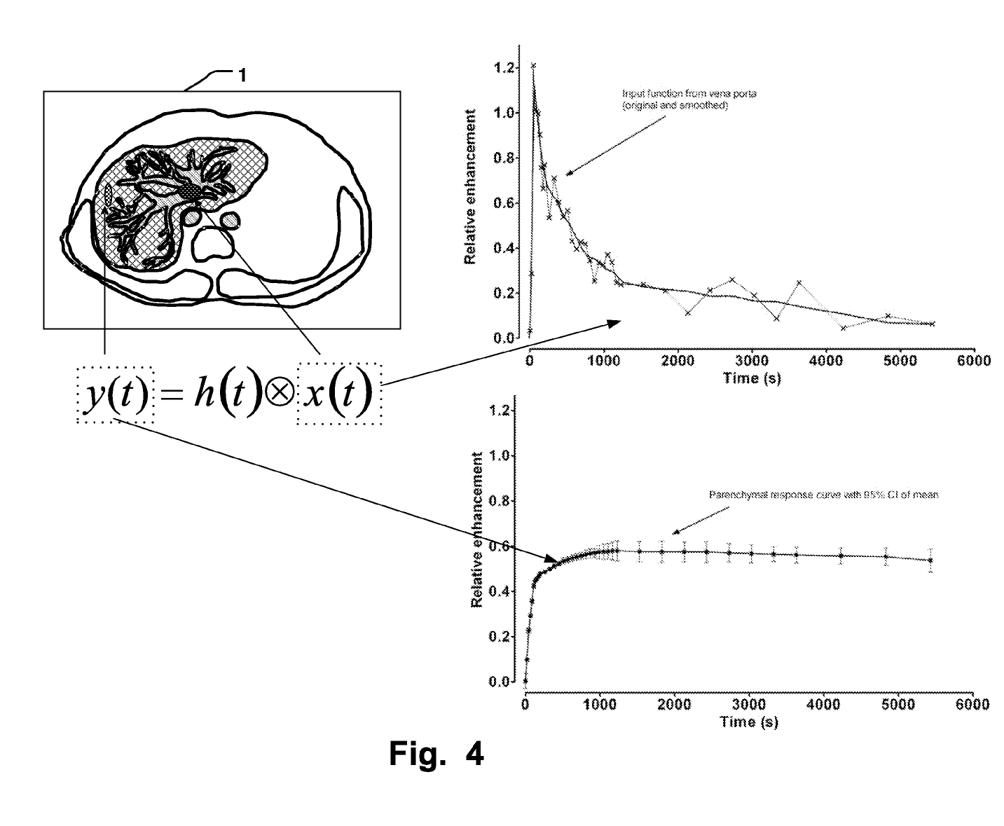

FIG. 4 is a schematic drawing illustrating the obtainment of a hepatic extraction curve.

The relative enhancement-over-time curve for the input function in the portal vein, and the parenchymal response function in liver segment V from one test subject are shown. The symbols (• and x) denote sample points. The parenchymal response curve is shown with a 95% confidence interval of the mean of the three ROIs placed in liver segment V. Both curves have been smoothed with a 7-point sliding window function.

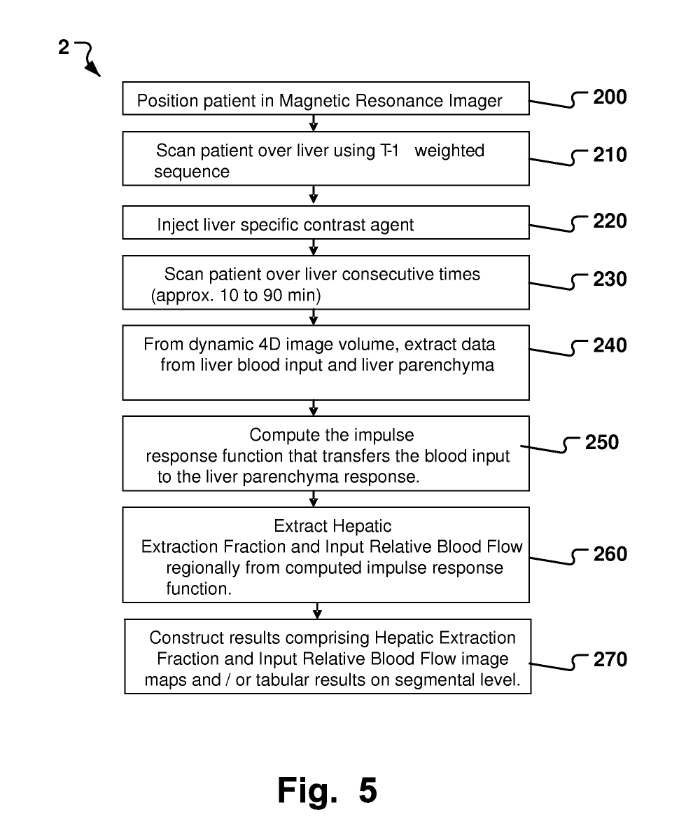

FIG. 5 is a flow chart illustrating a method 2 comprising an embodiment. A patient is positioned in an Magnetic Resonance Imager in step 200. The patient is scanned over the liver in step 210, using a T-1 weighted sequence providing 3D patient data comprising anatomical data for the liver and connected structures and organs.

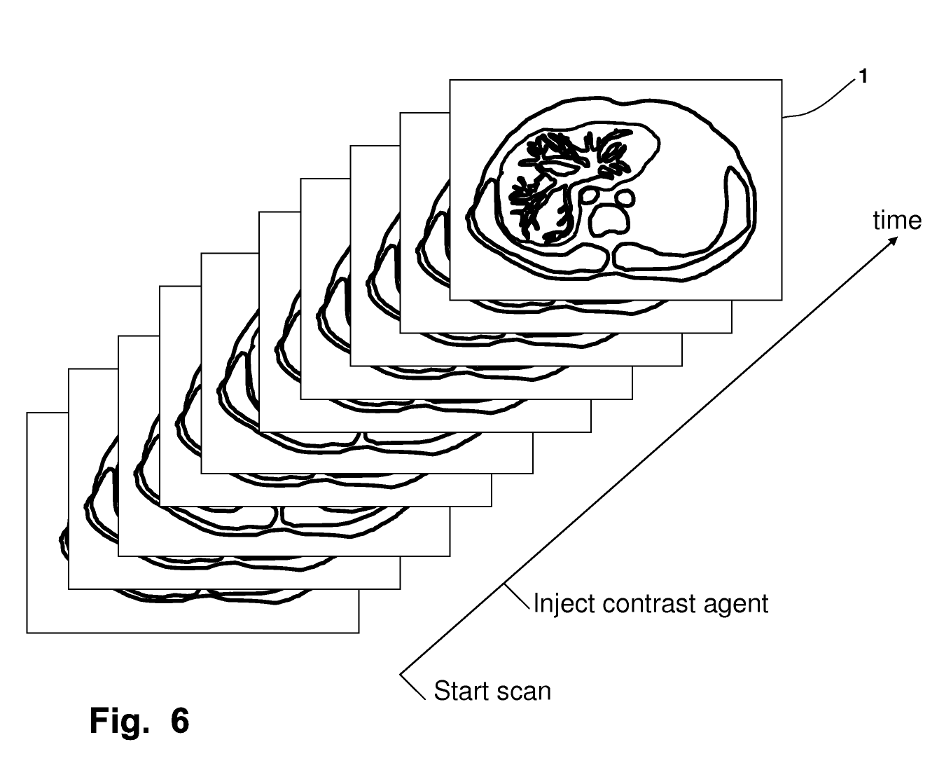

Then a liver specific contrast agent is injected into the blood flow of the patient in step 220. The patient is scanned over the liver by means of the Magnetic Resonance Imager consecutive times, during approximately 10 to 90 minutes, as illustrated in step 230. During each scan a new 3D data set is acquired, providing a four-dimensional (4D) data set, i.e. data for the temporal changes in the 3D volume are provided. The 4D data set is also called dynamic 4D image volume. This is illustrated in FIG. 6.

In the illustrated step 240 data is extracted from the dynamic 4D image volume for the liver blood input and the liver parenchyma, for instance using a method described in more detail below. The method may be computer implemented.

The impulse response function that transfers the blood input to the liver parenchyma response is calculated in step 250. This is for instance implemented by a suitable computer program. The calculation may be done as illustrated in FIG. 7, providing the Impulse Response function, also called Hepatic Extraction curve in Matrix form.

In step 260 the Hepatic Extraction Fraction and the input Relative Blood Flow are extracted regionally from the calculated impulse response function, providing data for further processing or analysis.

In step 270 the data from step 260 is used for providing Hepatic Extraction Fraction and the input Relative Blood Flow image maps and/or tabular results on a segmental level, or a sub-segmental level, down to a voxel level.

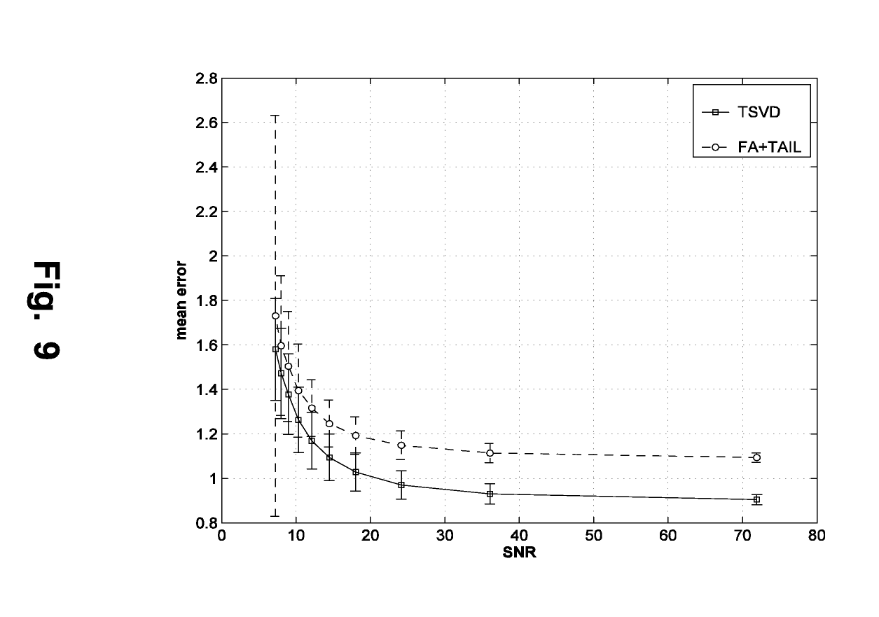

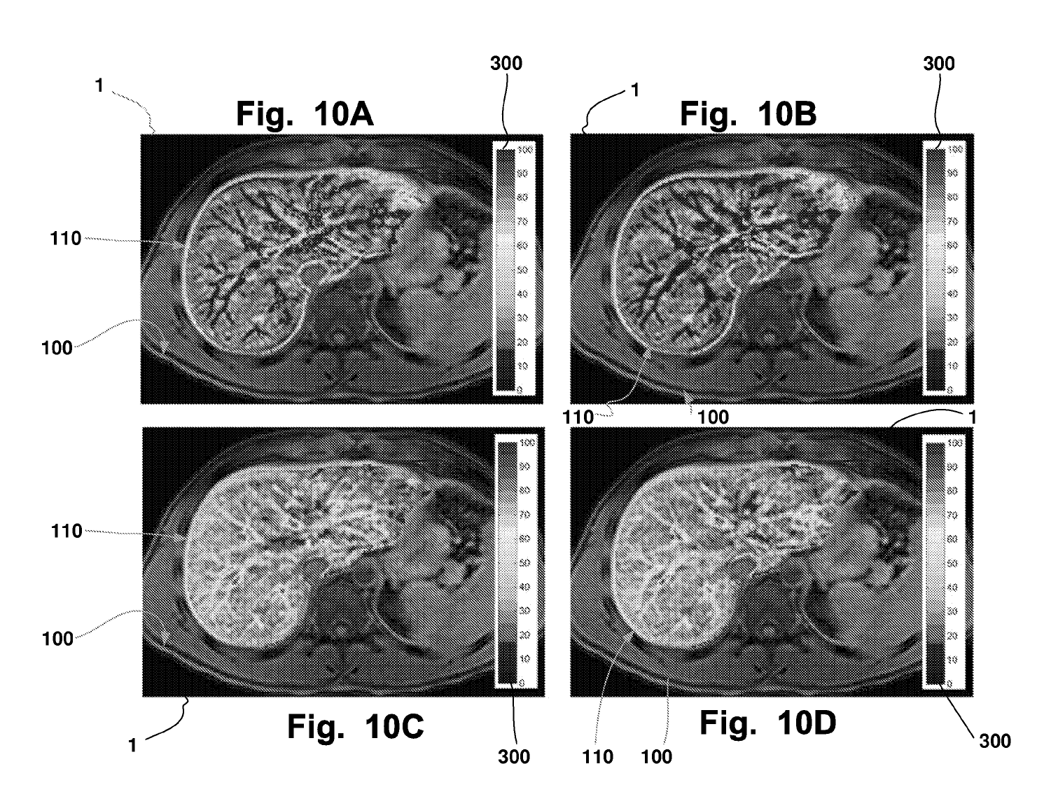

For instance in FIGS. 10A to 10D images are shown based on data acquired by DHCE-MRI and subsequent image processing based on different calculation methods. FIGS. 10A and 10C show parametric maps of HEF and irBF (inside 110) calculated with the TSVD DA respectively. FIGS. 10B and 10C show parametric maps of HEF and irBF calculated with FA DA, respectively. The parametric maps are color coded according to the color code table 300. The anatomical situation in the background (here in image 1 inside abdomen 100) is shown in black and white in order to clearly differentiate the illustrated organ function (HEF and irBF) in a segment of the organ, e.g. at a specific voxel.

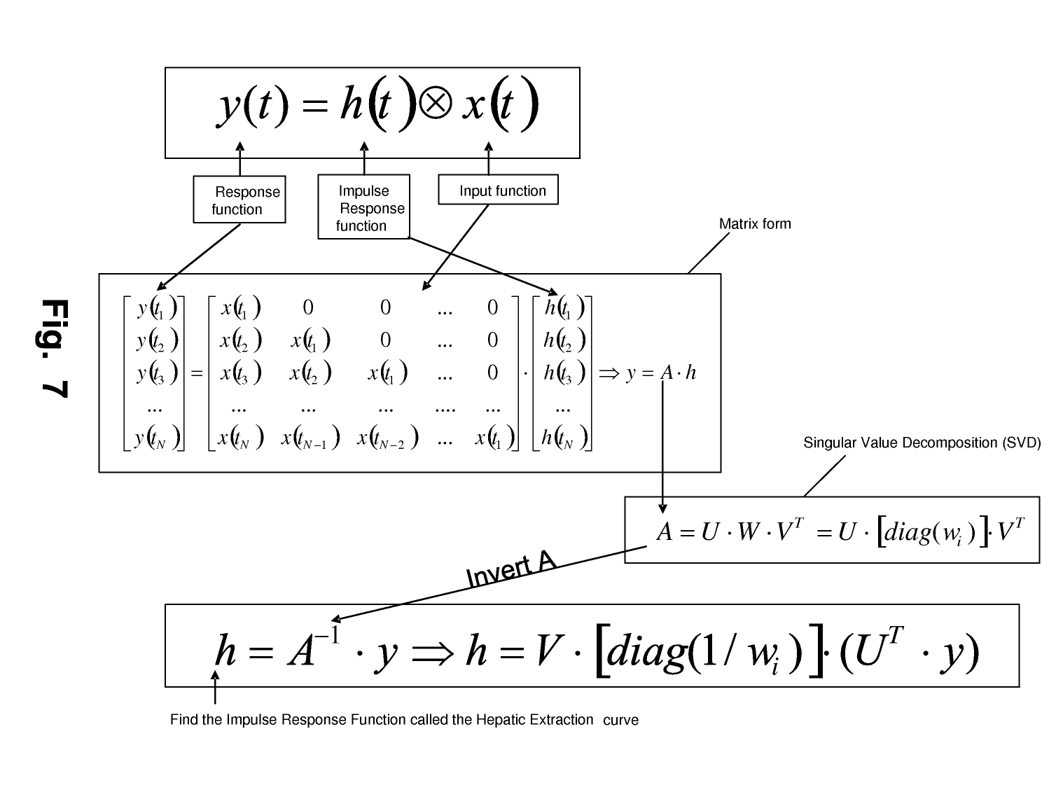

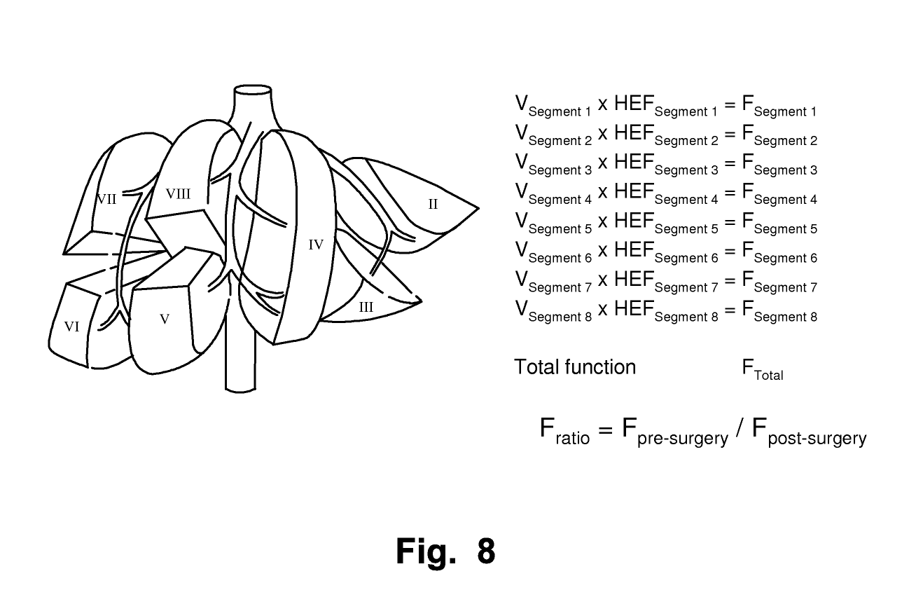

FIG. 8 is a schematic illustration of a sectionized hepatic function assessment.

The liver may be divided into eight segments (illustrated as I to VIII—Segment to Segment₈—SI to SVII) all functioning as separate organs with its own venous blood supply and billary excretion paths. HEF may thus be calculated for each voxel (x,y,z) throughout the liver. The liver volume may be obtained using computer-based segmentation and/or object identification, e.g. based on image intensity or Hounsfield grey values. Liver volume may be further divided into anatomic liver segments using semi-automatic computer software based on liver anatomy landmarks. A virtual function measure for the entire liver on a segmental or sub-segmental level may be obtained by multiplying the HEF with its corresponding volume.

$${V}_{Segment 1} \times {HEF}_{Segment 1} = {F}_{Segment 1}$$

$${V}_{Segment 2} \times {HEF}_{Segment 2} = {F}_{Segment 2}$$

$${V}_{Segment 3} \times {HEF}_{Segment 3} = {F}_{Segment 3}$$

$${V}_{Segment 4} \times {HEF}_{Segment 4} = {F}_{Segment 4}$$

$${V}_{Segment 5} \times {HEF}_{Segment 5} = {F}_{Segment 5}$$

$${V}_{Segment 6} \times {HEF}_{Segment 6} = {F}_{Segment 6}$$

$${V}_{Segment 7} \times {HEF}_{Segment 7} = {F}_{Segment 7}$$

$$\underline{{V}_{Segment 8} \times {HEF}_{Segment 8} = {F}_{Segment 8}}$$

$$Total function = sum , {F}_{Total}$$

If the liver function and/or the liver volume is altered by for example surgery or drug treatment, a new functional measure can be obtained using this technique. This change becomes a fraction.

Fᵣₐₜᵢₒ=F_{pre-surgery}/F_{post-surgery}

The ratio Fᵣₐₜᵢₒ is a ratio of the function before and after a treatment and may be applied to both drug treatment and surgery.

In addition, or alternatively, a virtual planning of the treatment is provided. For instance, a surgical removal of at least a portion of at least one segment of an organ may be virtually planned. The organ function after removal may be determined by the total function of the remaining segments. The surgeon thus is provided with valuable information if the estimated organ function after surgical removal of a portion thereof, still will be sufficient. Real surgery based on the virtual surgical planning may thus be adapted to the result thereof.

The provided measurement of blood flow may be used for the virtual planning. The input relative blood flow to different liver parts per volume segment may be determined. This is for instance clinically relevant for a heavily vascularized tumor. It is of interest to check for blood flow in a sub volume or the entire volume of the regarded 4D volume. The virtual planning includes even consideration of blood flow, e.g. when planning treatment with necrosis inducing pharmaceutical agents, such as Glivec®. During virtual planning the effect of such an agent may be defined for a region of a vascularised tumor. The virtual planning may thus provide a measure of the total liver function after the drug treatment.

In another example, a treatment with chemotherapy may be virtually planned in a computer implemented method of virtual planning of drug treatment. Thus a measure to interrupt or change the treatment is provided before the actual treatment, which is advantageous for the patient and also with regard to costs.

An example of assessing the feasibility to calculate HEF as a marker of hepatocyte function on a segmental level using dynamic Gd-EOB-DTPA-enhanced MRI is given below. The Fourier-based calculation method is compared with truncated SVD (TSVD) for deconvolutional analysis.

Further, examples of assessing HEF, irBF, pharmacokinetic transfer constants and semi-quantitative dynamic parameters in patients with primary biliary cirrhosis (PBC) and primary sclerosing cholangitis (PSC) using dynamic Gd-EOB-DTPA-enhanced MRI are given below.

Deconvolutional Analysis

Mathematically, the response function of an organ, in the embodiment the liver, can be described as a convolution between the impulse response and the input function,

y(t)=h(t) [symbol] x(t) [1]

where y(t) is the response function, h(t) the impulse function and x(t) the input function. The true liver function is characterized by the impulse function. FIG. 2A shows that the response y(t) equals the impulse function x(t), if the input function is ideal. Our input function consists of the injected tracer which will be dispersed over time due to recirculation. Therefore, our input function is not ideal and will greatly affect the response function y(t) as shown in FIG. 2B. The response function y(t) and the input function x(t) can be measured, but h(t) is unknown. However, with knowledge of the input and response functions, the impulse function can be estimated, either by Fourier analysis (FA), or matrix inversion. FA, described as

$$h ( t ) = {FT}^{- 1} \{ \frac{FT \{ y ( t ) \}}{FT \{ x ( t ) \}} \} , \qquad [ 2 ]$$

where FT is the Fourier transform and FT⁻¹ the inverse Fourier transform, has the advantage of being straightforward, but suffers from high-frequency artefacts resulting from the abrupt end points of x(t) and y(t). To avoid this abrupt end of data, a smooth appended curve can be added to the end of x(t) and y(t) to bring these curves back to zero. This is generally done by appending a cosine function from 0 to π/2 with the initial height of the last point of x(t) and y(t). It should be noted that two Fourier transformations have to be performed for each voxel. This is computationally extremely demanding, especially when having large data sets, such as the present patient 4D data sets. Historically, resolution of the imaging modalities is increasing with new developments thereof, whereby voxel size is minimized and the number of voxels increased. This trend further leads to an increased computational burden in the future, making clinically acceptable calculation times with sufficiently precise results even less feasible in the future.

However, by formulating the convolution in equation 1 into matrix form, the equation may according to an embodiment instead be solved by matrix inversion, using SVD as shown below, and as illustrated in FIG. 4:

$$[ \begin{matrix}y ( {t}_{1} ) \\ y ( {t}_{2} ) \\ y ( {t}_{3} ) \\ … \\ y ( {t}_{N} )\end{matrix} ] = [ \begin{matrix}x ( {t}_{1} ) & 0 & 0 & … & 0 \\ x ( {t}_{2} ) & x ( {t}_{1} ) & 0 & … & 0 \\ x ( {t}_{3} ) & x ( {t}_{2} ) & x ( {t}_{1} ) & … & 0 \\ … & … & … & … & … \\ x ( {t}_{N} ) & x ( {t}_{N - 1} ) & x ( {t}_{N - 2} ) & … & x ( {t}_{1} )\end{matrix} ] \cdot [ \begin{matrix}h ( {t}_{1} ) \\ h ( {t}_{2} ) \\ h ( {t}_{3} ) \\ … \\ h ( {t}_{N} )\end{matrix} ] ⇒ y = A \cdot h . \qquad [ 3.1 ]$$

Since A is a square matrix it will divide into SVD as,

A=U·W·V^{T}=U·[diag(wᵢ)]·V^{T} [3.2],

where U and V are orthogonal (i.e. their inverses equal their transposes) and W is diagonal with the elements wᵢ such as

w₁≧w₂≧ . . . w_{N}≧0 [3.3].

h(t) is solved by matrix inversion:

h=A⁻¹·y [symbol] h=V·[diag(1/wᵢ)]·(U^{T}·y) [3.4].

This is much less computationally burdensome than the above-mentioned two Fourier transformations necessary for each voxel. A global matrix is only calculated once and then available for all voxels.

If one or more of the wᵢ are zero or close to zero, the matrix inversion becomes ill-conditioned. Hence noise in the data becomes magnified in the least square solution (i.e. Eq 3.4) and makes the result of no practical value. One solution to this problem is the principle of regularization, or more specifically truncated SVD (TSVD). In TSVD the threshold, c ranging from 0 to 1, was defined as n(1−c), where n is the total number of singular values and c the threshold. For singular values smaller than this cut-off, 1/wi is not computed, but instead replaced by zero.

Important notes concerning deconvolution are the computational efficiency and the amount of data needed (i.e. the length of the 4D time resolved image data set). When comparing FA with TSVD-based deconvolution they are approximately equally fast for single voxel or ROI calculations. But for multi voxel deconvolution (i.e. calculation of parametric maps), SVD is superior with regard to efficiency since the matrix inversion seen in FIG. 7 only has to be computed once and then applied to all voxels of interest. On the contrary, the full DA must be performed for all voxels of interest using FA.

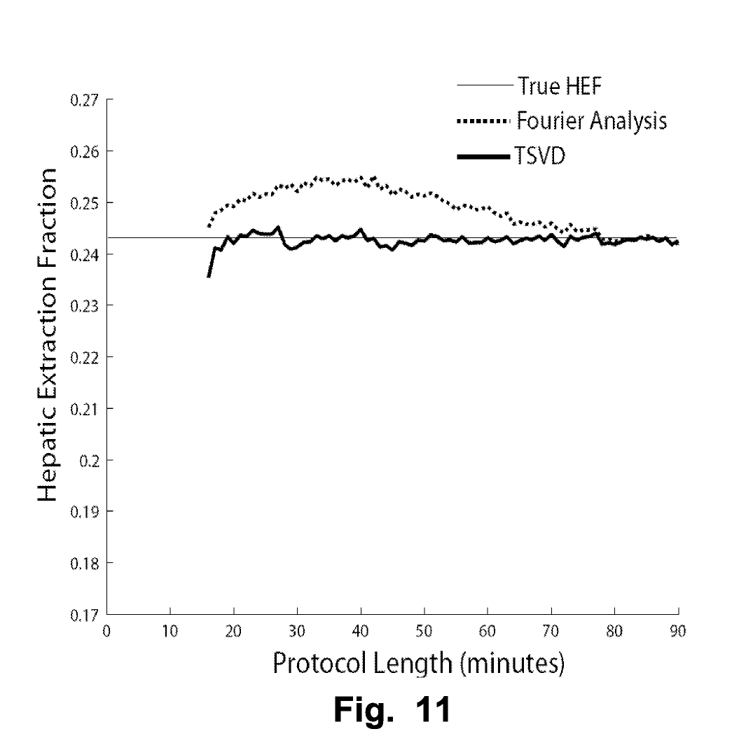

Moreover, the amount of data acquired is strictly limited by the length of the 4D imaging protocol. A simulation on protocol length using an ideal input and response function constructed from the average input and response function from 20 healthy volunteers shows that SVD-based deconvolution computes the same HEF value even though the protocol length is shortened. Scan protocols as short as 25 minutes were used to successfully calculate HEF. The result of this simulation is illustrated in FIG. 11. As seen in FIG. 11, FA DA overestimates HEF as the protocol becomes shorter.

FIG. 9 is a graph illustrating a mean error and error bars for such different simulated calculation methods.

Deconvolution Simulations

We performed a numerical simulation comparing the FA with appended tail (FA+tail) and TSVD. Ideal input and impulse functions were constructed from two gamma variate functions. Curve shapes were constructed to be as similar as possible to those measured in vivo. These two curves were then convoluted to find the response function, as shown in equation 1. Different amounts of normal distributed noise were applied to the response and input function respectively, to simulate different SNR levels. DA was then applied using the two different techniques. The appended tail in the FA+Tail technique was set to be three times the length of the simulation data. Truncation threshold in the TSVD technique was fixed at 0.07. Simulations were performed 1000 times for each SNR level and the standard deviation of the results using FA+tail was compared to the results obtained with TSVD using the variance ratio test.

Hepatic extraction fraction (HEF) and relative blood flow (RBF)

Deconvoluted liver response curves were analyzed with respect to HEF and RBF. HEF was described by Brown et al using Tc-99-disofenin scintigraphy as a measurement of hepatic extraction efficiency, and could be understood as the percentage of tracer that would have been extracted if the tracer had been injected directly into the afferent blood supply of the liver without subsequent recirculation. FIG. 3 shows a typical impulse response from liver parenchyma (hepatic extraction (HE) curve) using Gd-EOB-DTPA. The HE curve can be divided into the vascular phase and the hepatocyte retention phase, which describes the hepatic extraction. We calculated HEF using a monoexponential fit to the HE-curve data points from 420 to 1800 seconds after the time of injection of the tracer. The starting point at 420 seconds was chosen after visual inspection of the deconvoluted HE curves. The fitted curve, the hepatic retention curve (HRC, a mono-exponentially decaying fitted curve), is then extrapolated back to the time of the vascular peak value, and HEF is defined as the ratio between the extrapolated HRC curve and the vascular peak of the HE curve (also shown in FIG. 3),

$$HEF = 100 \cdot \frac{HRC ( t )}{{HE}_{max} ( t )} . \qquad [ 4 ]$$

RBF, giving a relative measurement of blood flow in the liver, is described as the initial peak value of the HE curve. RBF values were normalized to the segment with the highest RBF, i.e. the segment with the highest RBF was set to 100%.

Image Analysis

The input function was defined by a region of interest (ROI) placed in the hilar part of the portal vein. Due to patient motion, the input function ROI was adjusted in each dynamic acquisition so the voxels represent portal vein blood. Liver response function curves were defined by placing three ROIs in each liver segment (I to VIII with segment IV divided into IVa and IVb). The relative enhancement over time of the voxels in the ROI was regarded as the parenchymal response function for that ROI. Data points were interpolated using equidistant spacing (60 s) over the 90-minute time period. FIG. 3 shows a typical input function and parenchymal response function with interpolated data points. Care was taken to as far as possible to exclude major blood vessels and visible bile ducts when the ROIs were placed. Segments were defined and nomenclature adhered to as proposed by Strasberg SM. Terminology of liver anatomy and liver resections: coming to grips with hepatic Babel. J Am Coll Surg 1997; 184(4):413-434, which is incorporated herein by reference in its entirety. However, other segmentations may be used in other embodiments.

HEF and RBF were calculated for each ROI both with TSVD and FA+tail using specific in-house software written in MATLAB® (Mathworks, Michigan, USA). Thus, each ROI yielded two values for HEF and RBF respectively. For TSVD, a static truncation threshold was set at c=0.07. A cosine function from 0 to π/2 with the initial height of the last point of x(t) and y(t) was added for the DA performed with FA, and the length of the tail was set to be three times the length of the total sampling period of 90 minutes.

Parametric maps of HEF and RBF were calculated using the same input function as used for the segmental ROIs, but with each hepatic voxel representing a response function. RBF was always normalized to each subject's largest RBF value and presented as a percentage. To minimize effects of noise, mainly due to patient motion, low pass filtering of data was used by applying a seven point sliding window filter in both the input and response function curves.

Relative contrast agent concentrations in input and response functions were calculated as the logarithmic ratio,

$$C ( t , ρ ) = ln ( \frac{S ( t , ρ )}{{S}_{0} ( ρ )} ) , \qquad [ 5 ]$$

where c(t, ρ) is the relative tracer concentration at time t in voxel ρ. S0(ρ) is the mean image intensity in voxel ρ from the pre-contrast images, i.e. baseline signal intensity. S(t, ρ) is the measured image intensity in voxel ρ at time t.

Pharmacokinetic Compartmental Modeling

In a compartment model the distribution of a substrate passing between different compartments over time is modeled. The model is based on first order kinematics i.e. the time derivative of the concentration is negatively proportional to the concentration of the substrate itself. If the model consists of only one compartment, the equation describing the system is a one-dimensional first order differential equation.

$$\frac{d c ( t )}{d t} = - k \cdot c ( t ) \qquad [ 6 ]$$

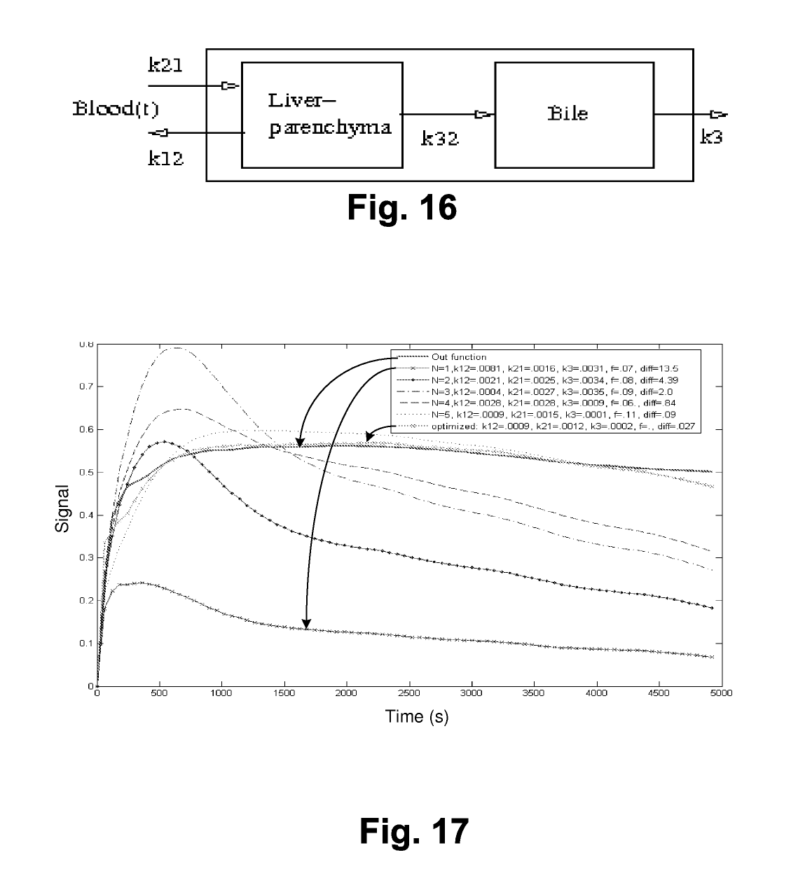

In pharmacokinetic modeling one can choose to include an arbitrary number of relevant compartments between which the substrate flows. When more than one compartment is modeled, the system equation becomes a system of differential equations. In our study we have used a three-compartment pharmacokinetic model described by Gambhir et al (J Nucl Med 1989; 30(9): 1507-1518) in a scintigraphy study using Tc-IDA as the tracer. The model is shown in FIG. 16, and can mathematically be described by

$$\begin{matrix}\frac{d v ( t )}{d t} = [ A ] v ( t ) + [ B ] u ( t ) y ( t ) = [ C ] v ( t ) + f \cdot {S}_{blood} ( t ) & [ 7 ] \\ [ A ] = [ \begin{matrix}- ( {k}_{3} + {k}_{12} ) & 0 \\ {k}_{3} & - {k}_{3}\end{matrix} ] & [ 8 ] \\ [ B ] = [ \begin{matrix}{k}_{21} \\ 0\end{matrix} ] [ C ] = [ \begin{matrix}1 & 1\end{matrix} ] & [ 9 ]\end{matrix}$$

In this system v(t)=(v₁(t), v₂(t)) is a vector representing the signal in the liver parenchyma and bile, y(t) is response function, and u(t) is the inflow to each volume. The term f·S_{blood}(t) represents the fraction f of the signal S_{blood}(t) from the blood pool, which adds to the signal from liver parenchyma. The parameters f and {k₁₂,k₂₁,k₃} (hereafter denoted kᵢⱼ) are the unknowns of the model. As shown in FIG. 16, k₂₁ denotes the flow-rate constant from blood to the liver compartment, k₁₂ denotes the back-flow from the liver compartment to the blood pool, the intrahepatic flux of bile from the hepatocytes to the bile canaliculi is described by k₃₂, and the flow from the intrahepatic to the extrahepatic bile compartment is described by the k₃ parameter. Mathematically the model is simplified by assuming that k₃₂ is equal to k₃, and also that there is no backflow from the bile canaliculi to the liver parenchyma.

In the special case when the input function is a pure bolus, or a Dirac-pulse, the response function y(t) will be identical to the input function x(t). The pure bolus dose assumption is an idealization however, but the response function y(t) can be calculated as a convolution between the impulse response h(t) and input function x(t), as discussed regarding the calculations of HEF. In the compartment model the impulse response function can analytically be described as the sum of two exponential functions containing the kᵢⱼ-parameters:

$$h ( t ) = {k}_{21} ( 1 - \frac{{k}_{3}}{{k}_{12}} ) {e}^{- ( {k}_{12} + {k}_{3} ) t} + {k}_{21} \frac{{k}_{3}}{{k}_{12}} {e}^{- {k}_{3} t} \qquad [ 10 ]$$

To arrive at an estimation of the model parameters, an iterative method was used. After assigning starting values to kᵢⱼ and f the impulse response function h(t) was estimated through Eq 10, and an output function yₒᵤₜ(t) was calculated through yₒᵤₜ(t)=h(t) [symbol] x(t)+f·x(t), using the input function x(t) from the portal vein as an estimate for the blood pool signal in Equation 7. The response function y(t) on the other hand was measured in a ROI in the liver parenchyma, and the parameters kᵢⱼ and f were determined by iteratively minimizing the squared difference diff=(y(t)−yₒᵤₜ(t))², see FIG. 17.

To increase the likelihood of finding the global minimum, a set of 10 randomized starting values was used; the values for kᵢⱼ and f were accepted only if the method converged to the same minimum more than 6 times out of 10.

The algorithm thus yielded five parameters, k₁₂, k₂₁, k₃, f and diff. The three transfer constants kᵢⱼ are defined in FIG. 16, the factor f defines the fraction of signal in the ROI originating from the blood pool (thus describing the perfusion in the ROI), and diff describes the goodness-of-fit for the converged response curve compared to those that were measured in the ROI.

Semi-Quantitative Analysis

The semi-quantitative parameters obtained directly from the parenchymal time-intensity curves were maximum relative signal intensity (Cₘₐₓ), time to maximum intensity (Tₘₐₓ), time from Tₘₐₓ to a five and ten percent decay in relative signal intensity (T₅ and T₁₀, respectively) and AUC from 0 to 5400 s. For some response curves either T₁₀, or both T₅ and T₁₀, were beyond the last measured time point, and no value was set. Tₘₐₓ, T₅ and T₁₀ were measured in seconds. Since the signal intensity half-time (T_{E}) with Gd-EOB-DTPA is much longer than the 90 minutes total scan time used in this study, T_{E} was estimated using through a bi-exponential fit, given by

g(t)=c₁·e^{-ln(2)·t/T}^{_{E}}−c₂·e^{-ln(2)·t/T}^{_{U}} [11]

where g(t) is the fitted curve and the fitting parameters c₂ and T_{U} describe contrast uptake, while c₁ and T_{E} describe the liver contrast excretion. Both T_{E} and T_{U} were calculated in minutes. The bi-exponential fit does not always converge if the whole response curve is included, and therefore t=240 s was empirically selected as the starting point for the fit.

Statistical Analysis

The mean HEF and RBF of the three segmental ROIs were regarded as the resulting HEF and RBF of that particular segment. Descriptive statistics (mean, standard deviation (SD), coefficient of variation (CV), median, maximum, minimum and range) were calculated for HEF and RBF with the two methods of DA respectively. The study yielded 180 paired observations of HEF and RBF (20 subjects with 9 segments each and each subject analyzed both with TSVD and FA+tail). The median HEF and RBF for the two methods of DA were compared using the nonparametric Wilcoxon matched pairs test, and the SD of the two methods was compared using the variance ratio test (also known as the F-test). A two-sided p-value less than 0.05 was regarded as significant. The Mann-Whitney U-test was used to compare non-paired data.

FIG. 12 is a schematic illustration of a system 1900 of an embodiment. The system 1900 is adapted for computer-based determining of a functional assessment of at least one organ having secretional or excretional functions, such as a liver and/or kidneys. The system comprises a unit for processing a four-dimensional (4D) image data set of said human comprising data for an assessment of said function of said at least one organ, wherein said 4D image data is acquired by an image modality processing a four-dimensional (4D) image data set of said human comprising data for an assessment of said liver function, wherein said 4D image data is acquired by an image modality. In an embodiment said unit for processing said 4D image data is arranged to perform a deconvolutional analysis (DA) comprising a matrix inversion using singular value decomposition (SVD) based on said 4D image data.

In an embodiment, the system 1900 is a computer-based system adapted to determine a function over time of at least one organ of a human is provided. The organ is an organ that has a secretional or excretional function, such as a liver and/or kidneys. The system comprises a processing unit configured to process a set of four-dimensional (4D) image data acquired by an image modality, and configured to determine a value of a parameter related to the function of the at least one organ per volume unit of the at least one organ based on the set of four-dimensional (4D) image data.

A diagnosis of a dysfunction of the organ is facilitated by a comparison of the determined value of the parameter with previously determined values of the parameters of a healthy population.

A medical workstation 1910 comprises the usual computer components like a central processing unit (CPU) 1920, memory, interfaces, etc. Moreover, it is equipped with appropriate software for processing data received from data input sources, such as data obtained from MRI scanning. Software may for instance be stored on a computer readable medium 1930 accessible by the medical workstation 1910. The computer readable medium 1930 may comprise the software in form of a computer program 1940 comprising suitable code segments 190. The medical workstation 1910 further comprises a monitor, for instance for the display of rendered visualizations, as well as suitable human interface devices, like a keyboard, mouse, etc., e.g. for manually fine-tuning an automatic planning otherwise provided by the software. The medical workstation may be part of the system 1900.

The computer program 1940 is storable on a computer readable medium, for processing by a computing device, such as CPU 1920 of medical workstation 1910, for determining a function over time of at least one secretional or excretional organ, such as a liver and/or kidneys of a human. The computer program comprises 1930 a plurality of code segments, comprising a first code segment 190 for determining a value of a parameter related to the function of the at least one organ per volume unit of the at least one organ based on processing of a set of four-dimensional (4D) image data of the human acquired by an image modality.

A diagnosis of a dysfunction of the organ is thus made possible in segments of the organ based on a comparison of the determined value of the parameter with previously determined values of the parameters of a healthy population. The parameter is for instance hepatic extraction fraction or input relative blood flow.

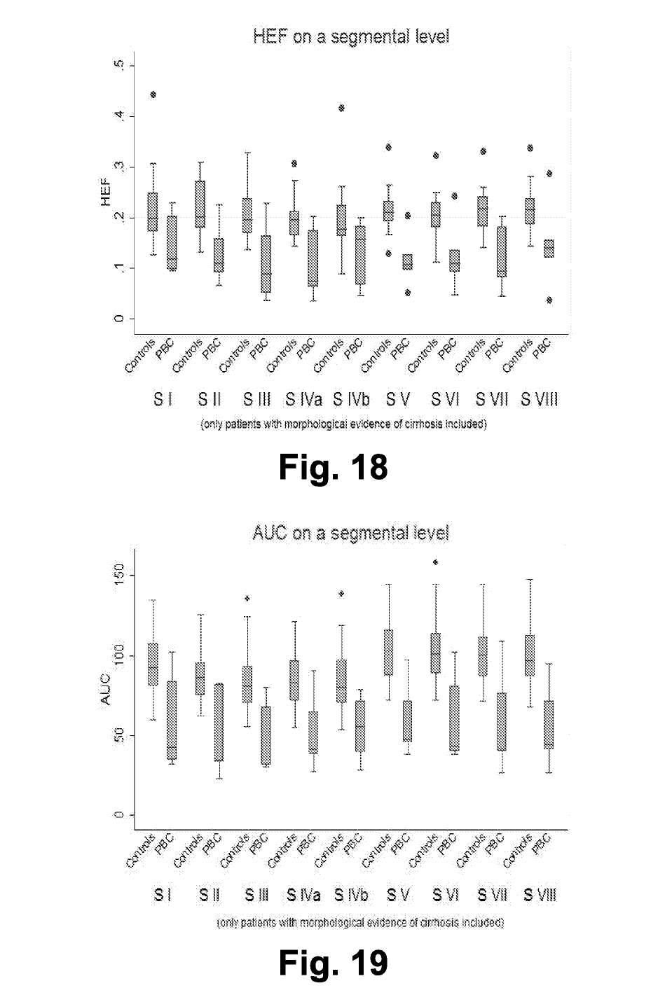

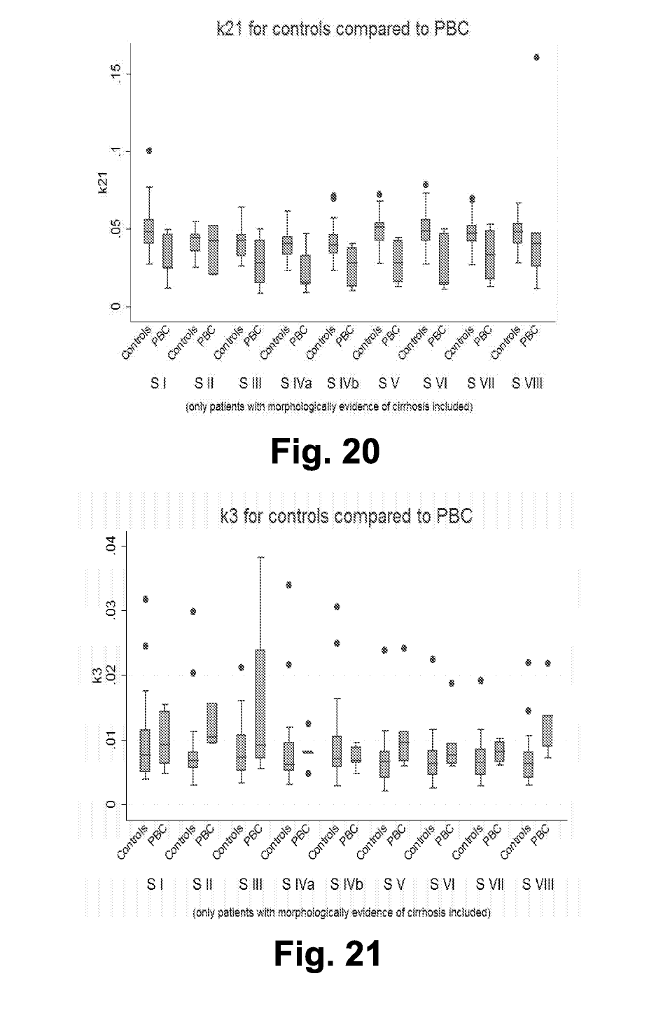

Examples of such comparisons with values from healthy populations are shown in FIGS. 18 to 25, and 25A and 26B, respectively.

A result of calculations or virtual planning described above may be provided to a user in a graphic user interface on the medical workstation 1910.

FIG. 13 is a schematic illustration of a computer program of an embodiment. The computer program is arranged for processing by a computing device functional assessment of at least one organ having secretional or excretional functions, such as a liver and/or kidneys, for processing by a computer is provided. The computer program may be embodied on a computer-readable medium and comprises a code segment 190 processing a four-dimensional (4D) image data set of said human comprising data for an assessment of said function of said at least one organ, wherein said 4D image data is acquired by an image modality, comprising performing a deconvolutional analysis (DA) comprising a matrix inversion using singular value decomposition (SVD) based on said 4D image data.

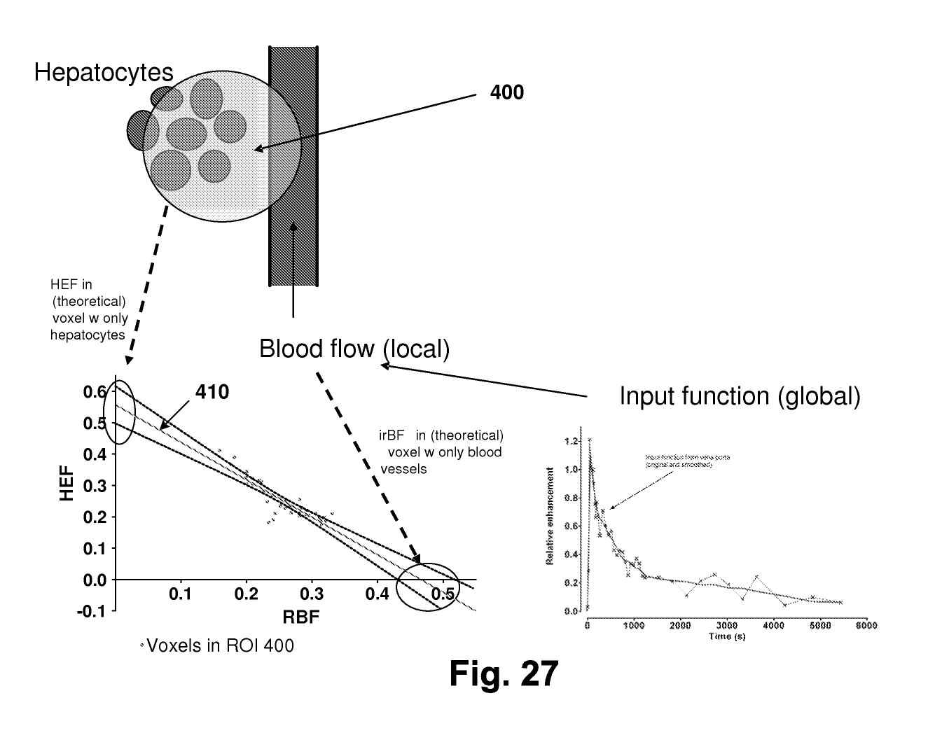

In FIG. 27, a ROI 400 with n voxels, all with different proportion of hepatocytes and blood vessels, is shown. HEF and irBF is calculated for each voxel and plotted. By linear regression, a straight line 410 is fitted to the data points acquired. In this manner local HEF and local irBF, with compensation for partial volume effects, is calculated and provided.

Some ailments, medical areas, procedures and/or organ diagnoses where the described method and/or system for the evaluation of segmental or sub-segmental liver function, liver perfusion and bile excretional function is of benefit for diagnosis, monitoring of disease progression, evaluation of treatment efficacy or adverse effects of treatment, include:

Hepatology:
Acute hepatitis
Chronic hepatitis
Primary Sclerosing Cholangitis
Primary Biliary Cirrhosis
Cystic fibrosis
Grading of Cirrhosis/Fibrosis and monitoring the progression of disease
Evaluation of the efficacy of choleretic drugs on bile flow in intrahepatic cholestasis
Evaluation of the impact of other forms of medical or immunological treatment on the liver
Obesity with NAFLD and NASH
Metabolic syndrome with impairment in liver function
Monitoring of liver function during surveillance for Hepatocellular Carcinoma among patients with Cirrhosis

Surgery:
Intrahepatic gallstone disease
Prediction of pre- and postoperative liver function for segmental liver surgery for colorectal cancer liver metastases and other primary and secondary tumours of the liver.
Evaluation of stent efficacy or EST (endoscopic sfincterotomy) on bile flow in obstructive jaundice
Evaluation of bile flow in malignant and benign tumours of the intra- and extrahepatic biliary tree
Evaluation of the patency and effectivity of all forms of hepatico-enteric bypasses.
Monitoring graft status in liver transplant patients

Oncology:
Chemotherapy-induced parenchymal injury (NASH, NAFLD, SOS)

### Example 1

### Subjects

T1-weighted Gd-EOB-DTPA-enhanced DHCE-MRI was performed on 20 healthy volunteers, 10 men and 10 women, ages ranging from 22 to 45 years. Routine serum liver function tests were performed at inclusion in the study. Test subjects had no history of hepato-biliary disease, previous hepato-biliary surgery or alcohol abuse.

Protocol

Data was collected using a Philips Intera 1.5T scanner (Best, Holland), with a Philips four-channel SENSE body coil. A T1-weighted 3D spoiled-gradient-echo pulse sequence (Repetition Time/Echo Time/Flip Angle 4.1 ms/2.0 ms/10 deg, Field Of View=415 mm, matrix resolution 256×192, 40 slices, slice thickness 10 mm and SENSE factor R=2) was used. The volume was imaged in a single breath hold at 41 different time points (12 seconds scan time per acquired volume). Three volumes were acquired pre-contrast for baseline calculations, followed by 38 volumes with step-wise increase in sampling intervals. The sampling density was chosen with respect to the subjects' physical capacity, data acquisition limitations and test substance dynamics. A dose of 0.1 ml/kg Gd-EOB-DTPA 0.25 mmol/ml was injected in the right anterior cubital vein, coinciding with the start of the fourth acquired volume. The contrast was injected using a power injector (Spectris MR injector System, Medrad, Pittsburgh), at an infusion rate of 2 ml per second, followed immediately by a bolus of 20 ml saline (NaCl 0.9%) at the same infusion rate.

Results

All subjects had normal serum liver function tests and no sign of renal insufficiency. The result of the simulation is shown in FIG. 9 as a SD comparison between the TSVD and the FA+Tail techniques. TSVD performs better than FA+Tail at higher SNR values. However, when data contain more noise, TSVD is more stable, with a much improved standard deviation.

Summary statistics for the HEF and RBF results with the two methods for DA are shown in Table 1.

TABLE 1

| (n = 20) | HEF:TSVD | HEF:FA + tail | | RBF:TSVD | RBF:FA + tail | |
|---|---|---|---|---|---|---|
| Mean | 0.215 | 0.217 | | 86.1% | 85.2% | |
| Median | 0.208 | 0.210 | (p = | 86.5% | 86.1% | (p = |
| | | | 0.524)¹ | | | 0.331)¹ |
| Min | 0.925 | 0.859 | | 58.5% | 55.6% | |
| Max | 0.436 | 0.440 | | 100% | 100% | |
| Range | 0.343 | 0.354 | | 41.5% | 44.0% | |
| SD | 0.0508 | 0.0548 | (p = | 10.5% | 10.6% | (p = |
| | | | 0.152)² | | | 0.458)² |
| CV | 23.6% | 25.3% | | 12.2% | 12.4% | |

¹Wilcoxon matched pairs test
²Variance ratio test

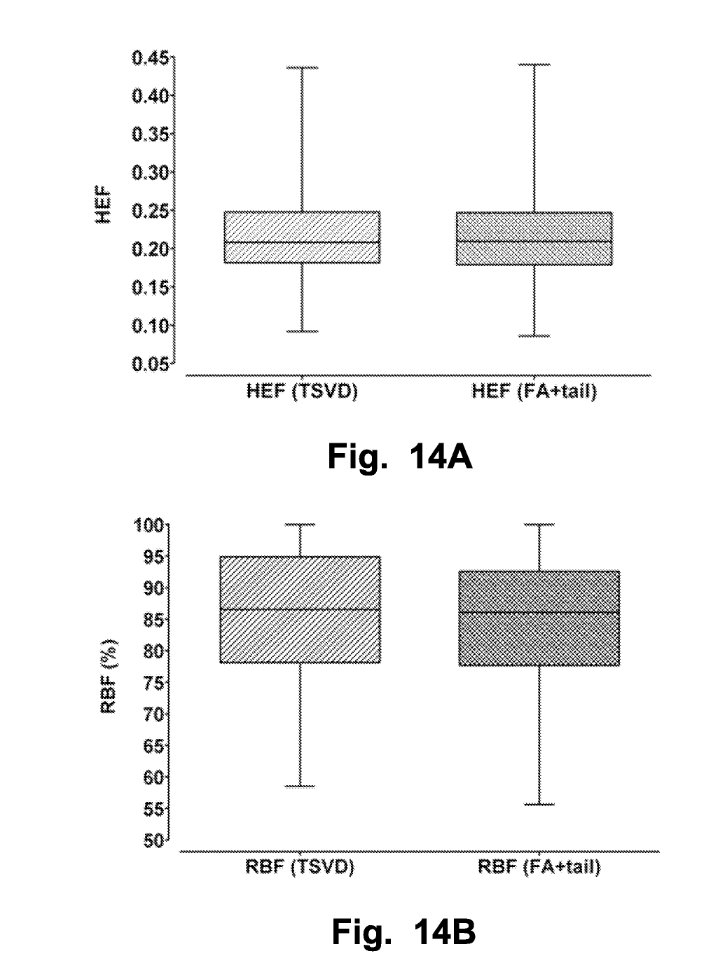

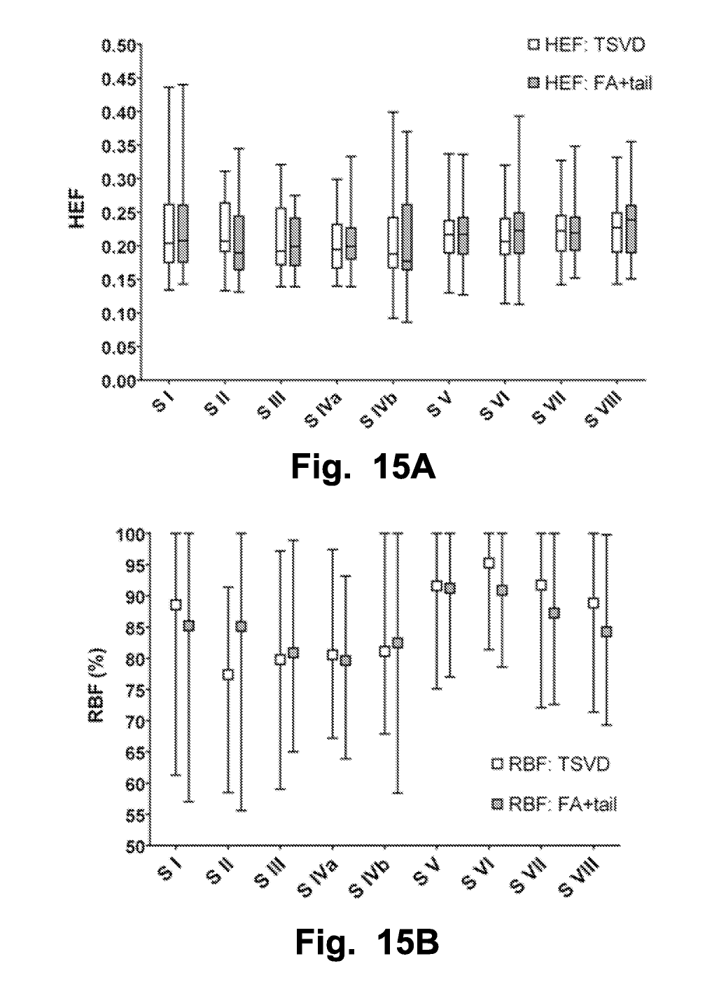

The HEF and RBF results from the 20 test subjects are presented graphically in FIGS. 14A and 14B, and the distribution of HEF and RBF on a segmental level is shown in FIGS. 15A and 15B. The mean ROI size was 31.9 (SD 21.6) voxels.

There was no significant difference in the overall results regarding HEF or RBF with the two methods (p=0.524 for HEF and p=0.331 for RBF), but TSVD yielded a smaller SD and a smaller CV, although the difference in SD was not significant (p=0.152 for HEF and 0.458 for RBF). There was a difference in the median HEF for the left and right hemilivers (0.196 for the left side and 0.218 for the right using TSVD and 0.194 vs. 0.224 using FA+tail for DA), but the difference was significant only when using the FA+tail technique (p=0.14 using TSVD vs. p=0.011 using FA+tail). There was also a difference in RBF between the left and right hemilivers with a significantly lower RBF in the left hemiliver, with a median RBF of 79.1% using TSVD and 81.2% using FA+tail. The corresponding values for the right side were 94.0% and 88.4% respectively. This difference was significant for both methods of DA (p<0.001 using the Mann-Whitney U-test).

Parametric maps of HEF (FIGS. 10A, B) and RBF (FIGS. 10C, D) for a slice above the horizontal inter-segmental plane of the liver in one test subject are shown in FIG. 10. By visual inspection, HEF seems to be homogenous throughout the slices. High values (i.e. close to 100%) in the parametric HEF-maps are results of voxels containing high levels of blood vessels and are not considered to reflect the hepatic function. Values above 100% were regarded as artefacts and excluded. All RBF values are scaled to the highest flow of each subject respectively.

In this example 1 it was found feasible to use DHCE-MRI with Gd-EOB-DTPA as tracer to assess HEF and RBF on a segmental level. It was also found that TSVD performs better than FA+tail for deconvolutional analysis in vivo. TSVD is less computationally demanding for the present area of application. Computer simulations also showed that DA with TSVD is less sensitive to noisy data with a significantly lower SD at lower SNR levels, thus TSVD should be the preferred choice for DA.

In scintigraphic studies on healthy subjects, HEF was around 100% when IDA-analogues with a near-total hepatic clearance were used. The mean HEF of slightly above 20% in this example 1 could very well reflect the known fact that Gd-EOB-DTPA has a lower hepatic affinity than the IDA-compounds, with a hepatic clearance of about 50%. Since Gd-EOB-DTPA has a different hepatic specificity, HEF may not be an optimal parameter to describe hepatocyte uptake using Gd-EOB-DTPA.

An interesting finding was the observed differences in HEF and RBF between the liver segments of the left and right liver lobes, see FIGS. 15A and 15B.

Intra-subject variation may in part be explained by motion artefacts over the acquisition period of 90 minutes. This in combination with partial volume effects of the ROIs may lead to noisy data with liver ROIs not necessarily reflecting liver parenchyma in the full dynamic volume. Motion artefacts in high resolution liver function tests should be minimized in order to increase data quality. On the other hand, intra-subject variation in HEF, may be a true phenomenon that has not been possible to detect with previous techniques.

In every study utilizing DA, the input function is vital for the results obtained. The liver has a dual vascular supply with venous inflow from the portal vein and arterial blood from the hepatic artery. We chose to use the enhancement-over-time curve from a ROI in the portal vein as the input function. The reasons for this were mainly physiological, since about 75% of the afferent blood flow to the liver emanates from the portal vein. Another reason is that the arterial input function has a very short peak, and with the temporal resolution in this example 1 we found empirically that we often missed the arterial peak resulting in worrying differences in maximum peak values in the arterial input function between our subjects. The portal peak is somewhat more dispersed in time and the differences in the peak values observed were much smaller. Three volumes per minute were acquired during the first three minutes.

In T1-weighted contrast enhanced DHCE-MRI, signal intensity is dependent on the T1-relaxation time. Higher concentrations of Gd-DTPA decrease the T1-relaxation time and increase image signal intensity. It has been shown that the relationship between image intensity and Gd-DTPA concentration is nonlinear for steady state MRI pulse sequences, such as the spoiled gradient echo used in this study. However, when T1-relaxation is within the range of 40 ms to 2600 ms, the MRI signal was shown to increase approximately exponentially with shortened T1-relaxation. All our measurements were estimated to be within this range, making equation 5a good approximation to relative contrast agent concentration.

### Example 2

### Investigation of Patients with Primary Biliary Cirrhosis (PBC)

Subjects

T₁-weighted Gd-EOB-DTPA-enhanced DHCE-MRI was performed on 20 healthy volunteers, 10 men and 10 women, and on patients with an established diagnosis of PBC. Routine serum liver function tests were performed at inclusion in the study on the healthy volunteers, and for the patients it was recorded from the most recent visit documented in their clinical charts. The healthy volunteers had no history of hepato-biliary disease, previous hepato-biliary surgery or alcohol abuse. All subjects were asked to be fasting for at least four hours prior to the examination. For each patient, relevant clinical data was documented and together with the results from the liver function tests they were used to calculate the CPS, Mayo risk score and MELD score.

MR Procedure

T₁-weighted Gd-EOB-DTPA-enhanced DHCE-MRI was performed using a Philips Intera 1.5T scanner (Best, Holland), with a Philips four-channel SENSE body coil according to the protocol in Example 1. Deconvolutional analysis was performed using truncated singular value decomposition (TSVD). HEF and irBF were calculated as described above. AUC was calculated quantitatively by assessing the area under the hepatic extraction curve from the peak value to 2700 seconds. Semi-quantitative parameters (SQP) and pharmacokinetic transfer constants were calculated as described above, and AUC was also semi-quantitatively calculated as the area under the parenchymal response curve from 0 to 5400 seconds. The Mann-Whitney U-test was used for test of significance and the significance level was set to α=0.5. All segments in every patient and controls yielded an observation and in the statistical analysis, all observations were regarded as independent observations, even if they originated from one individual. Thus, the study yielded 180 observations for each of the abovementioned parameters from the controls, and 108 observations from PBC patients.

Results

12 patients (of a planned total of 20 patients), (1 male, 11 female) have been included in the study. Patient characteristics, results of serum liver function tests (LFTs) and relevant clinical information are presented in Table 2.

TABLE 2
Patient characteristics

| | Patients | Controls | |
|---|---|---|---|
| | (n = 12) | (n = 20) | |
| Gender (m/f, n) | 1/11 | 10/10 | p < 0.05** |
| Age (yrs) | 62.8 | 33.2 | p < 0.05** |
| Bilirubin | 14 | 12.6 | p = 0.73** |
| Albumin | 35.5 | 42 | p < 0.05** |
| Creatinine | 67 | 83 | p < 0.05** |
| PK (INR) | 1.01 | 1.06 | p = 0.34** |
| Alk phos | 3.52 | 1.04 | p < 0.05** |
| Alat | 0.74 | 0.41 | p < 0.05** |
| AST | 0.77 | 0.32 | p < 0.05** |
| MELD | 7.48 | na | |
| Child-Pugh | 5.67 | na | |
| MayoScore | 5.46 | na | |
| Urso treatment | 1/11 | na | |
| (yes/no) | | | |
| Cirrhosis | 5/7 | na | |
| (yes/no)*** | | | |
| Ascites (yes/no) | 1/11 | na | |
| Edema (yes/no) | 0/12 | na | |
| Diuretics | 2/10 | na | |
| (yes/no) | | | |

*= proportionality test,
**= Student t-test,
***= Parenchyma judged cirrhotic on MR images

The patients with PBC were generally older than the controls, and the gender distribution was different, as would be expected. There was no significant difference regarding PK or bilirubin levels between the two groups, but albumin levels were significantly lower among PBC patients. AST, ALAT and alkaline phosphatase were all significantly higher among patients. The results of the quantitative parameters are shown in Table 3, and the results for the semi-quantitative parameters are shown in Table 4.

TABLE 3
Results for quantitative parameters obtained using DHCE-
MRI. Values are presented as the median value of the 9
segmental values for each patient. As a reference. the
median value for the 20 healthy controls is also presented.

| | HEF | irBF | AUC | k₂₁ | k₁₂ | k₃ | f | diff |
|---|---|---|---|---|---|---|---|---|
| Patient 1 | 0.152 | 0.283 | 87 | 0.054 | 0.042 | 0.012 | 0.354 | 0.049 |
| Patient 2 | 0.163 | 0.255 | 95 | 0.047 | 0.021 | 0.007 | 0.339 | 0.050 |
| Patient 3 | 0.163 | 0.357 | 117 | 0.058 | 0.020 | 0.008 | 0.495 | 0.089 |
| Patient 4 | 0.225 | 0.245 | 77 | 0.045 | 0.034 | 0.009 | 0.317 | 0.088 |
| Patient 5 | 0.302 | 0.229 | 91 | 0.049 | 0.027 | 0.009 | 0.309 | 0.065 |
| Patient 6 | 0.150 | 0.278 | 59 | 0.041 | 0.049 | 0.011 | 0.302 | 0.022 |
| Patient 7 | 0.093 | 0.218 | 42 | 0.022 | 0.048 | 0.011 | 0.308 | 0.032 |
| Patient 8 | 0.226 | 0.246 | 129 | 0.048 | 0.006 | 0.003 | 0.400 | 0.068 |
| Patient 9 | 0.199 | 0.269 | 100 | 0.057 | 0.034 | 0.009 | 0.370 | 0.076 |
| Patient | 0.155 | 0.276 | 84 | 0.035 | 0.017 | 0.006 | 0.448 | 0.101 |
| 10 | | | | | | | | |
| Patient | 0.106 | 0.232 | 28 | 0.013 | 0.018 | 0.006 | 0.354 | 0.057 |
| 11 | | | | | | | | |
| Patient | 0.047 | 0.241 | 38 | 0.013 | 0.012 | 0.014 | 0.313 | 0.023 |
| 12 | | | | | | | | |
| Median | 0.161 | 0.258 | 82 | 0.044 | 0.025 | 0.009 | 0.352 | 0.062 |
| for PBC | | | | | | | | |
| patients | | | | | | | | |
| (n = 108) | | | | | | | | |
| Median | 0.201 | 0.240 | 91 | 0.045 | 0.017 | 0.007 | 0.331 | 0.060 |
| for | | | | | | | | |
| controls | | | | | | | | |
| (n = 180) | | | | | | | | |
| p-value* | p < 0.01 | p < 0.01 | p < 0.01 | p = 0.21 | p < 0.01 | p < 0.01 | p < 0.01 | p = 0.07 |

*Mann-Whitney U-test

TABLE 4
Results for semi-quantitative parameters obtained using DHCE-MRI.
Values are presented as the median value of the 9
segmental values for each patient.
As a reference, the median value for the 20 healthy
controls is also presented.

| | Cmax | Tmax | T₅ | T₁₀ | T_{E} | AUC |
|---|---|---|---|---|---|---|
| Patient 1 | 0.612 | 3100 | 940 | 1440 | 1281 | 2734 |
| Patient 2 | 0.583 | 2900 | 1460 | 1830 | 3780 | 2858 |
| Patient 3 | 0.614 | 2100 | 1090 | 1680 | 1591 | 3024 |
| Patient 4 | 0.593 | 2200 | 2440 | 2280 | 4791 | 3030 |
| Patient 5 | 0.607 | 2900 | 1980 | 2040 | 6961 | 3076 |
| Patient 6 | 0.487 | 4000 | 1490 | 3030 | 4241 | 2485 |
| Patient 7 | 0.392 | 2600 | 1300 | 1950 | 1180 | 1980 |
| Patient 8 | 0.641 | 3600 | 2040 | 2880 | 8011 | 3216 |
| Patient 9 | 0.577 | 1360 | 1480 | 2460 | 2130 | 2663 |
| Patient 10 | 0.561 | 2400 | 1540 | 2480 | 2481 | 2822 |
| Patient 11 | 0.386 | 480 | 810 | 1800 | 1141 | 1735 |
| Patient 12 | 0.317 | 200 | 240 | 940 | 2830 | 1392 |
| Median for | 0.552 | 2460 | 1440 | 1920 | 2850 | 2708 |
| PBC patients | | | | | | |
| (n = 108) | | | | | | |
| Median for | 0.534 | 2000 | 1600 | 2295 | 3130 | 2665 |
| controls (n = 180) | | | | | | |
| p-value* | p = 0.70 | p < 0.01 | p < 0.05 | p < 0.001 | p = 0.33 | p = 0.58 |

HEF was significantly lower and irBF was significantly higher among PBC patients, but the uptake transfer k₂₁ was not different compared to controls. The transfer rate constants k₁₂ and k₃ were higher among patients than controls, as was the factor f that designates the fraction of blood in the ROI. There was no significant difference regarding the goodness-of-fit parameter diff between the groups. Regarding the semi-quantitative parameters, there were no significant differences regarding maximum intensity (Cmax), excretion half-time (T_{E}) or area-under-curve (AUC). Time to maximum intensity (Tmax) was significantly longer among PBC patients, but the excretion parameters T₅ and T₁₀ were shorter. HEF and AUC (quantitatively calculated) were lower with increasing Child-score.

In this study, we found a significantly lower HEF in patients with PBC as would be expected, and the difference seems to increase with increasing severity of disease, as described above. It is known that liver cirrhosis leads to an increase in arterial blood flow and a reduction in portal flow. Maybe an increased arterial peak in cirrhotic liver parenchyma can explain the differences in irBF noticed in this study. Since PBC leads to an obliteration of the fine bile ducts, one would expect the time to maximum enhancement to be longer since the gadolinium tracer accumulates over a longer time in the hepatocyte. When we only look at the patients with a morphological evidence of liver cirrhosis, we find an even greater difference compared to the healthy controls. In FIG. 18-21 this is shown on a segmental level where the quantitative parameters are compared between the healthy controls and the 5 patients that had signs of cirrhosis on the MR images obtained in the study. One would expect the k₃ parameter to be lower among PBC patients than controls but this seems not to be the case. The study parameters used seem to be able to detect deterioration in parenchymal function with regard to uptake differences, but not to quantify differences in biliary excretion.

### Example 3

### Investigation of Patients with Primary Sclerosing Cholangitis (PSC)

Subjects

T₁-weighted Gd-EOB-DTPA-enhanced MRI was performed on 20 healthy volunteers, 10 men and 10 women, and on patients with an established diagnosis of PSC. Routine serum liver function tests were performed at inclusion in the study on the healthy volunteers, and for the patients it was recorded from the most recent visit documented in their clinical charts. The healthy volunteers had no history of hepato-biliary disease, previous hepato-biliary surgery or alcohol abuse. All subjects were asked to be fasting for at least four hours prior to the examination. For each patient, relevant clinical data was documented and together with the results from the liver function tests they were used to calculate the CPS, Mayo risk score and MELD score.

MR Procedure

T₁-weighted Gd-EOB-DTPA-enhanced MRI was performed using a Philips Intera 1.5T scanner (Best, Holland), with a Philips four-channel SENSE body coil according to the protocol as outlined in Example 1. Deconvolutional analysis was performed using truncated singular value decomposition (TSVD). HEF and irBF were calculated as described above. AUC was calculated quantitatively by assessing the area under the hepatic extraction curve from the peak value to 2700 seconds. Semi-quantitative parameters (SQP) and pharmacokinetic transfer constants were calculated as described above, and AUC was also semi-quantitatively calculated as the area under the parenchymal response curve from 0 to 5400 seconds. The Mann-Whitney U-test was used for test of significance and the significance level was set to α=0.5. All segments in every patient and controls yielded an observation and in the statistical analysis, all observations were regarded as independent observations, even if they originated from one individual. Thus, the study yielded 180 observations for each of the abovementioned parameters from the controls, and 108 observations from PSC patients.

Results

12 patients (of a planned 20 patients), have been included in the study. The demographic and clinical parameters of included patients and controls are summarized in Table 5.

TABLE 5
Patient characteristics

| | Patients | Controls | |
|---|---|---|---|
| | (n = 12) | (n = 20) | |
| Sex (m/f) | 7/5 | 10/10 | p = 0.0850* |
| Age | 42.1 | 33.2 | p < 0.05** |
| Bilirubin | 10.9 | 12.6 | p = 0.52** |
| Albumin | 36 | 42 | p < 0.01** |
| Creatinine | 69 | 83 | p < 0.05** |
| PK | 1.09 | 1.06 | p = 0.57** |
| Alkaline Phosphatase | 3.66 | 1.04 | p < 0.05** |
| Alat | 1.63 | 0.41 | p < 0.05** |
| AST | 1.14 | 0.32 | p < 0.01** |
| MELD | 7.54 | na | |
| Child | 5.55 | na | |
| Mayo | −0.05 | na | |
| Urso treatment (yes/no) | 8/4 | na | |
| Abnormal parenchyma? | 16% | na | |
| (Percent of segments) | | | |
| Ascites (yes/no) | 0/12 | na | |
| EST (yes/no) | 1/11 | na | |
| History of variceal | 0/12 | na | |
| bleed? (yes/no) | | | |

*= proportionality test.
**= Student t-test.
***= Parenchyma judged abnormal on MR images

The patients with PSC were generally older than the controls, and the gender distribution was different. There was no significant difference regarding PK or bilirubin levels between the two groups, but albumin levels were significantly lower among PSC patients. AST, ALAT and alkaline phosphatase were all significantly higher among patients. The results of the quantitative and semi-quantitative parameters are shown in Table 6 and 7 respectively.

TABLE 6
Results for quantitative parameters obtained using DHCE-
MRI. Values are presented as the median value of the 9
segmental values for each patient. As a reference, the
median value for the 20 healthy controls is also presented.

| | HEF | irBF | AUC | k₂₁ | k₁₂ | k₃ | f | diff |
|---|---|---|---|---|---|---|---|---|
| Patient 1 | 0.136 | 0.252 | 100 | 0.037 | 0.006 | 0.006 | 0.318 | 0.045 |
| Patient 2 | 0.240 | 0.299 | 87 | 0.034 | 0.015 | 0.006 | 0.553 | 0.254 |
| Patient 3 | 0.158 | 0.314 | 101 | 0.043 | 0.016 | 0.006 | 0.407 | 0.047 |
| Patient 4 | 0.213 | 0.201 | 63 | 0.027 | 0.017 | 0.006 | 0.278 | 0.094 |
| Patient 5 | 0.221 | 0.275 | 117 | 0.070 | 0.028 | 0.010 | 0.309 | 0.036 |
| Patient 6 | 0.307 | 0.242 | 94 | 0.083 | 0.071 | 0.015 | 0.220 | 0.023 |
| Patient 7 | 0.140 | 0.220 | 61 | 0.027 | 0.020 | 0.007 | 0.299 | 0.036 |
| Patient 8 | 0.220 | 0.220 | 75 | 0.049 | 0.046 | 0.012 | 0.288 | 0.089 |
| Patient 9 | 0.159 | 0.281 | 97 | 0.033 | 0.010 | 0.004 | 0.448 | 0.129 |
| Patient 10 | 0.136 | 0.231 | 58 | 0.066 | 0.091 | 0.019 | 0.225 | 0.026 |
| Patient 11 | 0.123 | 0.267 | 58 | 0.042 | 0.053 | 0.012 | 0.310 | 0.044 |
| Patient 12 | 0.174 | 0.225 | 71 | 0.046 | 0.045 | 0.024 | 0.241 | 0.014 |
| Median for | 0.176 | 0.254 | 78 | 0.042 | 0.023 | 0.009 | 0.308 | 0.049 |
| PSC | | | | | | | | |
| patients | | | | | | | | |
| (segments | | | | | | | | |
| n = 108) | | | | | | | | |
| Median for | 0.201 | 0.240 | 91 | 0.045 | 0.017 | 0.007 | 0.331 | 0.060 |
| controls | | | | | | | | |
| (segments | | | | | | | | |
| n = 180) | | | | | | | | |
| p-value* | p < 0.001 | p < 0.05 | p < 0.001 | p = 0.399 | p < 0.010 | p < 0.001 | p = 0.380 | p < 0.05 |

*Mann-Whitney U-test

TABLE 7
Results for semi-quantitative parameters obtained using DHCE-MRI.
Values are presented as the median value of the 9 segmental
values for each patient. As a reference, the median value for the
20 healthy controls is also presented.

| | Cₘₐₓ | Tₘₐₓ | T₅ | T₁₀ | T_{E} | AUC |
|---|---|---|---|---|---|---|
| Patient 1 | 0.532 | 2200 | 1340 | 2460 | 341 | 2599 |
| Patient 2 | 0.669 | 3800 | 2140 | 3540 | 452 | 3373 |
| Patient 3 | 0.644 | 3400 | 1720 | 1980 | 1427 | 3191 |
| Patient 4 | 0.448 | 2500 | 980 | 1560 | 110 | 2164 |
| Patient 5 | 0.603 | 2800 | 1620 | 1950 | 184 | 3014 |
| Patient 6 | 0.564 | 1620 | 940 | 1720 | 102 | 2573 |
| Patient 7 | 0.424 | 1860 | 1420 | 2645 | 244 | 2171 |
| Patient 8 | 0.680 | 1800 | 1380 | 1840 | 211 | 3366 |
| Patient 9 | 0.668 | 2880 | 1170 | 2340 | 251 | 3344 |
| Patient 10 | 0.494 | 1280 | 1400 | 2600 | 203 | 2113 |
| Patient 11 | 0.560 | 2200 | 1250 | 2050 | 147 | 2695 |
| Patient 12 | 0.470 | 1680 | 1500 | 2490 | 128 | 2317 |
| Median for PSC | 0.559 | 2260 | 1340 | 2050 | 204 | 2693 |
| patients | | | | | | |
| (segments | | | | | | |
| n = 108) | | | | | | |
| Median for | 0.534 | 2000 | 1600 | 2295 | 313 | 2665 |
| controls | | | | | | |
| (segments | | | | | | |
| n = 180) | | | | | | |
| p-value* | p = 0.052 | p < 0.05 | p < 0.01 | p < 0.05 | p < 0.01 | p = 0.29 |

HEF was significantly lower and irBF was significantly higher among PSC patients, and the quantitatively calculated AUC was significantly smaller among patients. The uptake transfer constant k₂₁ did not differ between groups. The transfer rate constants k₁₂ and k₃ were higher among patients than controls, but the factor f that designates the fraction of blood in the ROI, did not differ. There was a significant difference regarding the goodness-of-fit parameter diff between the groups with a generally better fit among patients. Regarding the semi-quantitative parameters, there were no significant differences regarding maximum intensity (Cmax), excretion half-time (TE) or area-under-curve (AUC). Time to maximum intensity (Tmax) was significantly longer among PSC patients, but the excretion parameters T5 and T10 were shorter.

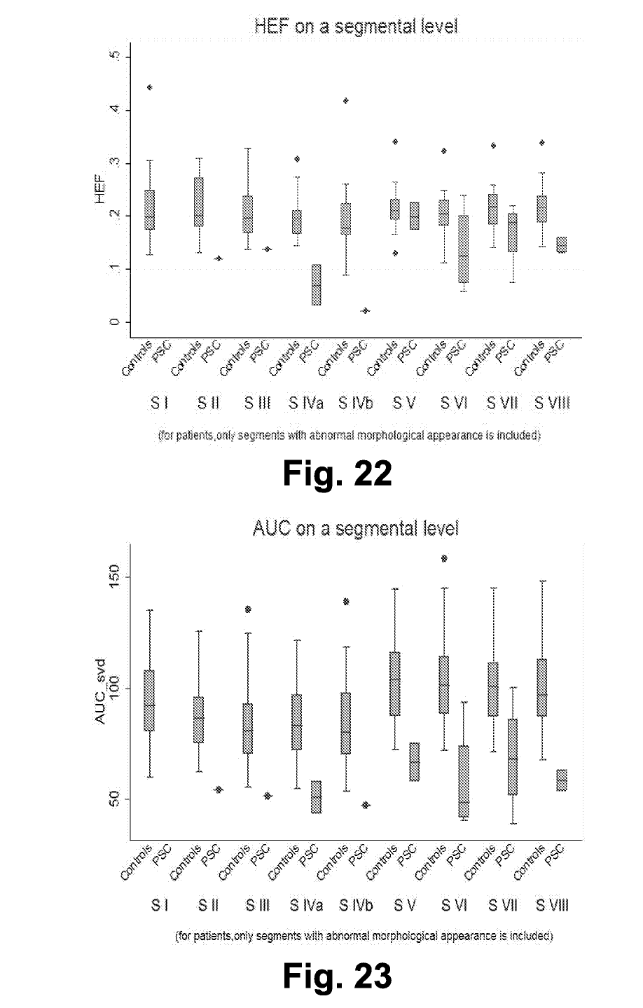

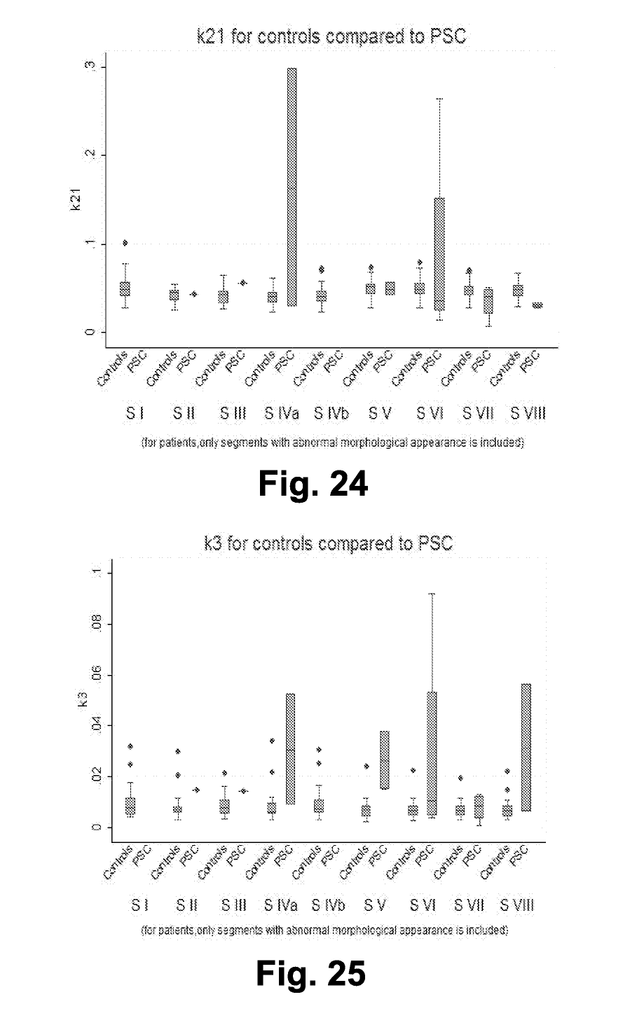

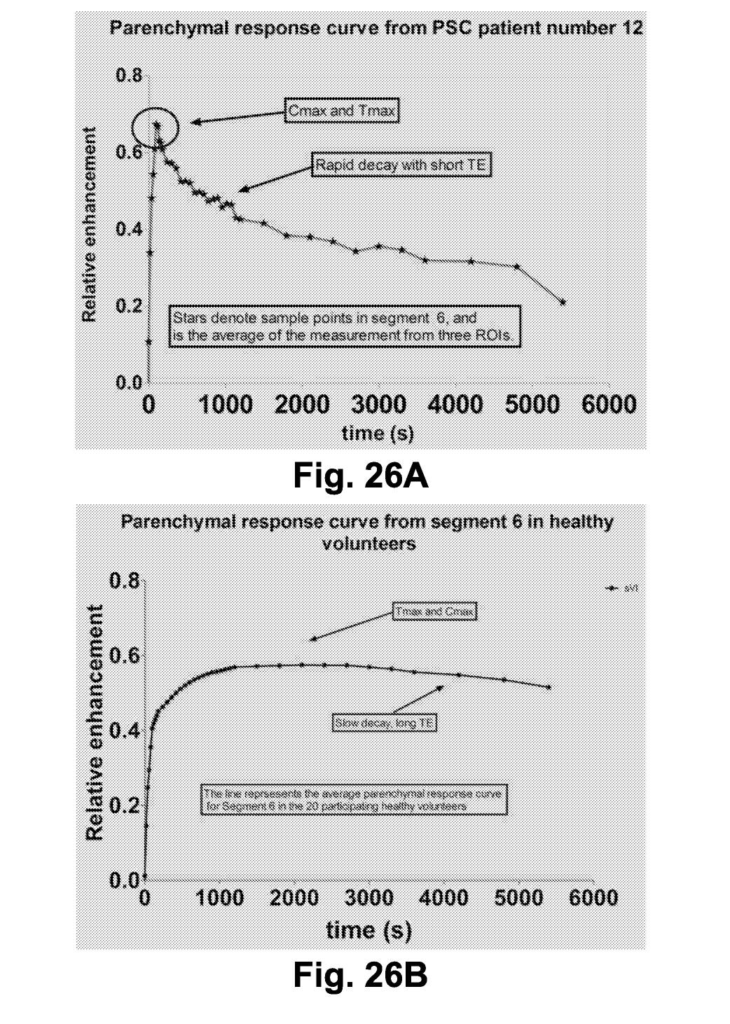

The patient population in this study had relatively mild disease with low MELD and Mayo-scores. Only one patient was Child B. Nevertheless, significant differences in hepatic uptake of the tracer indicating differences in parenchymal function could be detected using T₁-weighted DHCE-MRI with Gd-EOB-DTPA. If HEF and AUC are plotted against the results of healthy volunteers, there seems to be a trend towards less uptake for the AUC parameter if the disease has a higher score, but this is not as evident for HEF. If we plot the results on a segmental level, and only plot segments with abnormal contrast-enhancement patterns, there is a notable difference in HEF and AUC between normal and abnormal parenchyma (FIGS. 22 and 23). The transfer-rate constants k₂₁ and k₃ seem to be higher in more severely affected parenchyma, but the explanation for this is obscure (FIGS. 24 and 25). An interesting finding was a small but significant hyperperfusion of the liver parenchyma among PSC patients as understood by the increase in irBF. Theoretically, this could be the result of an ongoing inflammatory process in the liver parenchyma, or possibly an arterialisation of a cirrotic or fibrotic parenchyma. Regarding Cmax, it did not differ between the groups and this could have several explanations. When visually inspecting a parenchymal response curve from a part of the parenchyma with an abnormal appearance, it is evident that it differs from the parenchymal response curve of the healthy volunteers (FIG. 26). Tmax is significantly higher among patients, but the excretion seems to be faster (Table 7) with a lower T₅ and T₁₀. Perhaps an explanation for this could be the choleretic effects of the ursodeoxycholic acid treatment that ⅔ of the patients were currently on at time of inclusion.

A further example is an implantation of a stent in the biliary duct in order to open an obstructed biliary duct. An evaluation of the stent efficacy is provided on a comparison of pre- and post-implantation organ function or bile flow.

In conclusion, a novel method and system are disclosed for the evaluation of hepatocyte function per volume on a segmental level. In embodiments DHCE-MRI, such as with dynamic Gd-EOB-DTPA enhanced MRI, is used in human healthy volunteers. Instead of using summary parameters, a mathematical model applying DA with both FA+tail and TSVD is presented. TSVD, being slightly less sensitive to noisy data than Fourier-based DA, is the preferred method for deconvolution of data obtained with DHCE MRI in liver function tests.

The method and/or system are also useful for enabling, providing or performing a virtual planning of treatments, as described further above.

The method and/or system may also be applied to other organs having secretional or excretional functions, such as for instance a placenta, a digestive system, or a pancreas.

The method and/or system may be applied to determine the function of several organs simultaneously. Distributions of functions between these organs may be calculated and further processed.

As will be appreciated by one skilled in the art, the present invention may be embodied as device, system, method or computer program product. Accordingly, the present invention may take the form of an entirely hardware embodiment, a software embodiment or an embodiment combining software and hardware aspects. Furthermore, the present invention may take the form of a computer program product on a computer-usable storage medium having computer-usable program code embodied in the medium. Any suitable computer readable medium may be utilized including hard disks, optical storage devices, transmission media such as those supporting the Internet or an intranet, or magnetic storage devices.

The present invention has been described above with reference to specific embodiments. However, other embodiments than the above described are equally possible within the scope of the invention. The scope of the invention is only limited by the appended patent claims.

1. A computer-based system (1900) adapted to determine a function over time of at least one organ of a human, said organ having a secretional or excretional function, such as a liver and/or kidneys, said system comprising
a processing unit configured to process a set of four-dimensional (4D) image data acquired by an image modality, and configured to determine a value of a parameter related to said function of said at least one organ per volume unit of said at least one organ based on said set of four-dimensional (4D) image data, whereby a diagnosis of a dysfunction of said organ is facilitated by a comparison of said determined value of said parameter with previously determined values of said parameters of a healthy population.

2. The system of claim 1, wherein said volume unit is at least one segment or at least one sub-segment, or a plurality of segments or a plurality of sub-segments, of said at least one organ and said processing unit is configured to process said 4D image data on a segmental or sub-segmental level of said at least one organ.

3. The system of claim 2, wherein said processing unit is configured to determine said function of said at least one organ based on a blood flow in said least one organ determined by said processing unit in said segment or sub-segment of said organ relative to an arterial blood flow into said at least one organ.

4. The system of claim 2, wherein said processing unit is configured to determine said function of said at least one organ comprises is configured to determine a blood flow in said segment or sub-segment of said at least one organ relative to a venous blood flow from said at least one organ, such as a liver, or kidneys, or a liver and kidneys.

5. The system of claim 2, wherein said at least one organ comprises a liver and said system is adapted to determine a function of said liver that comprises an input relative Blood Flow (irBF) of said liver based on a blood flow in said at least one segment or sub-segment, or said plurality of segments or sub-segments, of said liver, determined by said processing unit, relative to an input blood flow to said liver.

6. The system of claim 1, wherein said at least one organ comprises a liver and said system is adapted to determine a function of said liver that comprises a Hepatic Extraction Fraction (HEF) of said liver in at least one segment or sub-segment, or a plurality of segments or sub-segments, of said liver.

7. The system of claim 6, wherein said processing unit is configured to determine said Hepatic Extraction Fraction (HEF) based on truncated singular value decomposition (TSVD) calculations.

8. The system according to claim 7, wherein said processing unit is configured to determine a parametric map for said HEF and/or irBF based on said TSVD calculations.

9. The system of claim 1, wherein said volume unit is determined in said 4D image data set.

10. The system of claim 1, wherein said image modality is a Magnetic Resonance Imaging (MRI) modality, and wherein said 4D image data set is an MRI image data set obtained by T1-weighted dynamic contrast enhanced (DCE) MRI, and wherein said 4D image data set is at least partly contrast enhanced by a contrast agent specific for said at least one organ.

11. The system of claim 10, wherein said contrast agent specific for said at least one organ is a hepatocyte-specific contrast agent, and said 4D image data set is an MRI image data set obtained by T1-weighted Dynamic Hepato Specific Contrast Enhanced (DHCE) Magnetic Resonance Imaging (MRI).

12. The system of claim 1, wherein said system is adapted to simultaneously determine both a liver and a renal function.

13. The system of claim 12, wherein said 4D image data set is an MRI image data set obtained by Dynamic Hepato-Renal Specific Contrast Enhanced (DHRCE) Magnetic Resonance Imaging (MRI).

14. The system of claim 11, wherein said hepatocyte-specific contrast agent is Gadolinium ethoxybenzyl diethylenetriaminepentaacetic acid (Gd-EOB-DTPA).

15. The system of claim 5, wherein said processing unit is configured to determine said input relative Blood Flow (irBF) based on truncated singular value decomposition (TSVD) calculations.

16. The system of claim 1, wherein said processing unit is configured to perform a deconvolutional analysis (DA) comprising a matrix inversion using singular value decomposition (SVD) based on said 4D image data.

17. The system of claim 1, wherein said volume unit is a voxel in said 4D data.

18. A computer program storable on a computer readable medium, for processing by a computing device for determining a function over time of at least one secretional or excretional organ, such as a liver and/or kidneys of a human,
said computer program comprising a plurality of code segments, comprising a first code segment for determining a value of a parameter related to said function of said at least one organ per volume unit of said at least one organ based on processing of a set of four-dimensional (4D) image data of said human acquired by an image modality, facilitating diagnosis of a dysfunction of said organ by a comparison of said determined value of said parameter with previously determined values of said parameters of a healthy population.

19. A computer-implemented method of determining a function over time of at least one secretional or excretional organ, such as a liver and/or kidneys of a human,
wherein said determining said function of said at least one organ comprises determining a value of a parameter related to said function of said at least one organ per volume unit of said at least one organ, and wherein determining said function is based on processing of a set of four-dimensional (4D) image data of said human acquired by an image modality, facilitating diagnosis of a dysfunction of said organ by a comparison of said determined value of said parameter with previously determined values of said parameters of a healthy population.

20. The method of claim 19, wherein said volume unit is at least one segment or at least one sub-segment, or a plurality of segments or a plurality of sub-segments, of said at least one organ and said processing said 4D image data is executed on a segmental or sub-segmental level of said at least one organ.

21-42. (canceled)
